(19) United States
(12) Patent Application Publication
(10) Pub. No.: US 2024/0344115 A1
(43) Pub. Date: Oct. 17, 2024

(54) METHODS AND COMPOSITIONS FOR QUANTIFYING IMMUNE CELL DNA

(71) Applicant: GUARDANT HEALTH, INC., Palo Alto, CA (US)

(72) Inventors: Yupeng HE, Alhambra, CA (US); Ariel JAIMOVICH, Redwood City, CA (US); Andrew KENNEDY, San Diego, CA (US); William J. GREENLEAF, Menlo Park, CA (US); Meromit SINGER, Menlo Park, CA (US); Emily Katherine TSANG, Mountain View, CA (US)

(21) Appl. No.: 18/470,187

(22) Filed: Sep. 19, 2023

Related U.S. Application Data
(63) Continuation of application No. PCT/US22/71365, filed on Mar. 25, 2022.
(60) Provisional application No. 63/166,200, filed on Mar. 25, 2021.

(51) Int. Cl. C12Q 1/6827; C12Q 1/6806; C12Q 1/6886

(52) U.S. Cl. CPC C12Q 1/6827; C12Q 1/6806; C12Q 1/6886; C12Q 2600/154

(57) ABSTRACT

Provided herein is a DNA analysis method for detecting and quantifying immune cell types from which the DNA originated. Provided herein are also methods for determining the likelihood that a subject has a disease or condition, such as cancer.

### CROSS-REFERENCE TO RELATED APPLICATIONS

This application is a continuation of International PCT Application No. PCT/US2022/071365 filed Mar. 22, 2022, which claims the benefit of priority of U.S. Provisional Application No. 63/166,200, filed Mar. 25, 2021, which is incorporated by reference herein in its entirety for any purpose.

### FIELD OF THE INVENTION

The present disclosure provides methods and compositions related to analyzing DNA, such as cell-free DNA, originally present in immune cells. In some embodiments, the DNA is from a subject having or suspected of having a disease or disorder, such as cancer. In some embodiments, the immune cell types from which the DNA originated are identified and quantified.

### INTRODUCTION AND SUMMARY

Invasive diagnostic procedures, including biopsies, are commonly used for detecting or diagnosing cancer, ulcers, liver diseases, infections, transplant rejections, and other diseases and disorders in which analysis of cells or tissue from a possible site of a malady are analyzed for relevant features. Detection of diseases and disorders based on analysis of body fluids (“liquid biopsies”), such as blood, is an intriguing alternative. A liquid biopsy is noninvasive, sometimes requiring only a blood draw. However, it has been challenging to develop accurate and sensitive methods for analyzing liquid biopsy material because the amount of nucleic acids released into body fluids is low and variable as is recovery of nucleic acids from such fluids in analyzable form. For example, detecting the presence of circulating tumor DNA (ctDNA) from early-stage cancer is difficult due to its low abundance.

An alternative or supplemental approach is to detect the signal linked to secondary effects of the presence of a disease such as cancer. One such signal of a secondary effect is the signal from the immune response to tumorigenesis. In the presence of tumor cells, immune cells proliferate, differentiate, and potentially turn over at a high rate than in a healthy subject. Such phenomena can result in increased shedding of immune cell DNA into the bloodstream Therefore, a secondary immune signal may be useful for detecting diseases or disorders, such as cancer, with improved sensitivity in at least some circumstances.

Furthermore, quantification of different blood cell types provides important information about a subject's overall health in addition to information about disease states. The ability to distinguish between different cell types, including closely related cell types, can be important for distinguishing between different types of diseases and disorders. A number of studies report that the cfDNA in plasma are primarily from blood cells (myeloid cells) in normal state. However, in a disease state, the cfDNA from immune cell types may be elevated, which can be used as an indicator of the disease. In a number of genomic regions, the DNA methylation signature in different immune cell types are distinguishable from myeloid cells and other immune cell types. Existing methods, such as complete blood counts and microarray-based profiling of differentially methylated regions of cfDNA allow detection of many cell types but do not discriminate between certain types of immune cells, e.g., naïve and activated lymphocytes, and therefore do not provide important information about the state of the subject's immune system.

The methods herein provide an approach to quantify the levels of circulating cell-free DNA (cfDNA) derived from different immune cell types based on DNA methylation data generated by partitioning DNA based on extent of methylation and next-generation DNA sequencing. In some embodiments, methods herein use genomic regions that are differentially methylated between specific immune cell types and other blood cells and methyl binding assays to profile the methylation status of cfDNA fragments from these regions, facilitating quantifying the presence of one or more specific immune cell types. Applications of this approach include cancer detection by detecting tumor-induced immune cell proliferation.

The methods herein can also provide combined information about other DNA variations and modifications, including but not limited to sequence variations.

The present disclosure aims to meet the need for improved analysis of DNA originating from different immune cell types, including rare immune cell types, such as activated and naïve lymphocytes. Improved differentiation of immune cell types over existing methods, such as complete blood count (CBC) or DNA methylation based methods that do not discriminate between immune cell types, allows for more accurate detection of disorders (diagnosis) and therefore improved treatments. Accordingly, the following exemplary embodiments are provided.

Embodiment 1. A method of analyzing cfDNA in a sample, the method comprising:
a) sequencing the cfDNA and determining methylation levels for an epigenetic target region set comprising a plurality of target regions comprising DNA sequences that are differentially methylated in a plurality of immune cell types; and
b) determining quantities of each of a plurality of immune cell types from which the cfDNA originated based on the methylation levels, wherein the plurality of immune cell types comprises
i. naïve and activated lymphocytes;
ii. monocytes and macrophages; or
iii. myelocytes, neutrophils, and eosinophils.

Embodiment 2. A method of analyzing cfDNA in a sample, the method comprising:
a) capturing at least an epigenetic target region set from the cfDNA or a subsample thereof, comprising contacting the cfDNA or subsample thereof with target-specific probes specific for the at least one epigenetic target region set, wherein the epigenetic target region set comprises target regions comprising DNA sequences that are differentially methylated in a plurality of immune cell types;
b) determining methylation levels for the target regions; and
c) determining quantities of each of the plurality of immune cell types from which the DNA originated, wherein the plurality of immune cell types comprises
i. naïve and activated lymphocytes;
ii. monocytes and macrophages; or
iii. myelocytes, neutrophils, and eosinophils.

Embodiment 3. A method of analyzing cfDNA in a sample, the method comprising:
a) sequencing the cfDNA and determining methylation levels for an epigenetic target region set comprising a plurality of hypomethylation variable target regions comprising DNA sequences that are differentially hypomethylated in a plurality of immune cell types; and
b) determining quantities of each of a plurality of immune cell types from which the cfDNA originated based on the methylation levels.

Embodiment 4. A method of analyzing cfDNA in a sample, the method comprising:
a) capturing at least an epigenetic target region set from the cfDNA or a subsample thereof, comprising contacting the cfDNA or subsample thereof with target-specific probes specific for the at least one epigenetic target region set, wherein the epigenetic target region set comprises hypomethylation variable target regions comprising DNA sequences that are differentially hypomethylated in a plurality of immune cell types;
b) determining methylation levels for the target regions; and
c) determining quantities of each of the plurality of immune cell types from which the DNA originated.

Embodiment 5. A method of analyzing DNA in a sample, the method comprising:
a) partitioning the sample into a plurality of subsamples by contacting the DNA with an agent that recognizes a modified cytosine in the DNA, the plurality comprising a first subsample and a second subsample, wherein the first subsample comprises DNA with the modified cytosine in a greater proportion than the second subsample;
b) sequencing DNA from one or more of the plurality of subsamples; and
c) detecting levels of DNA sequences to determine quantities of each of a plurality of immune cell types from which the DNA originated, wherein the plurality of immune cell types comprises
i. naïve and activated lymphocytes;
ii. monocytes and macrophages; or
iii. myelocytes, neutrophils, and eosinophils.

Embodiment 6. A method of analyzing DNA in a sample, the method comprising:
a) partitioning the sample into a plurality of subsamples by contacting the DNA with an agent that recognizes a modified cytosine in the DNA, the plurality comprising a first subsample and a second subsample, wherein the first subsample comprises DNA with the modified cytosine in a greater proportion than the second subsample;
b) capturing at least an epigenetic target region set of DNA from at least one of the first and second subsamples, comprising contacting the DNA with target-specific probes specific for the at least one epigenetic target region set, wherein the target regions of the epigenetic target region set comprise DNA sequences that are differentially methylated in a plurality of immune cell types, wherein the plurality of immune cell types comprises
iv. naïve and activated lymphocytes;
V. monocytes and macrophages; or
vi. myelocytes, neutrophils, and eosinophils,
vii. thereby providing captured DNA; and
c) sequencing the captured DNA and determining levels of each of a plurality of immune cell types from which the DNA originated.

Embodiment 7. A method of analyzing DNA in a sample, the method comprising:
a) partitioning the sample into a plurality of subsamples by contacting the DNA with an agent that recognizes a modified cytosine in the DNA, the plurality comprising a first subsample and a second subsample, wherein the first subsample comprises DNA with the modified cytosine in a greater proportion than the second subsample;
b) capturing at least an epigenetic target region set of DNA from at least one of the first and second subsamples, wherein at least one epigenetic target region set comprises a hypomethylation variable target region set, thereby providing captured DNA;
c) sequencing the captured DNA; and
d) detecting the levels of captured DNA sequences and determining levels of each of a plurality of immune cell types from which the DNA originated.

Embodiment 8. A method of analyzing DNA in a sample, the method comprising:
a) partitioning the sample into a plurality of subsamples by contacting the DNA with an agent that recognizes a modified cytosine in the DNA, the plurality comprising a first subsample and a second subsample, wherein the first subsample comprises DNA with the modified cytosine in a greater proportion than the second subsample;
b) capturing at least an epigenetic target region set of DNA from at least one of the first and second subsamples, comprising contacting the DNA with target-specific probes specific for the at least one epigenetic target region set, wherein the target regions of the epigenetic target region set comprise DNA sequences that are hypomethylated in a plurality of immune cell types, thereby providing captured DNA; and
c) sequencing the captured DNA.

Embodiment 9. The method of embodiment 5 or 7, wherein the method comprises capturing at least an epigenetic target region set of DNA from at least one of the first and second subsamples, wherein the target regions of the epigenetic target region set comprise DNA sequences that are differentially methylated in a plurality of immune cell types, and wherein the capturing is performed prior to the sequencing.

Embodiment 10. The method of any one of the preceding embodiments, wherein the epigenetic target region set comprises a hypermethylation variable target region set and a hypomethylation variable target region set.

Embodiment 11. The method of any one of the preceding embodiments, wherein the plurality of immune cell types comprises naïve and activated lymphocytes.

Embodiment 12. The method of the immediately preceding embodiment, wherein the plurality of immune cell types comprises naïve T cells, naïve B cells, effector BCD4 T cells, effector CD8 T cells, Treg cells, plasma cells, and memory cells.

Embodiment 13. The method of the immediately preceding embodiment, wherein the effector CD4 T cells comprise effector memory CD4 T cells and central memory CD4 T cells, and wherein the effector CD8 T cells comprise effector memory CD8 T cells and central memory CD8 T cells.

Embodiment 14. The method of any one of the preceding embodiments, wherein the plurality of immune cell types comprises monocytes and macrophages.

Embodiment 15. The method of any one of the preceding embodiments, wherein the plurality of immune cell types comprises myelocytes, neutrophils, and eosinophils.

Embodiment 16. The method of any one of the immediately preceding embodiment, wherein the plurality of immune cell types comprises metamyelocytes.

Embodiment 17. The method of any one of the preceding embodiments, wherein the plurality of immune cell types comprises natural killer (NK) cells.

Embodiment 18. The method of any one of the preceding embodiments, wherein the levels of each of the plurality of immune cell types are determined relative to levels of total blood cells.

Embodiment 19. The method of any one of the preceding embodiments, wherein the sample is a blood sample.

Embodiment 20. The method of any one of the preceding embodiments, wherein the sample is a plasma sample.

Embodiment 21. The method of any one of the preceding embodiments, wherein the sample is obtained from a tissue sample.

Embodiment 22. The method of the immediately preceding embodiment, wherein the tissue sample is a biopsy, a fine needle aspirate, or a formalin-fixed paraffin-embedded tissue sample.

Embodiment 23. The method of any one of the preceding embodiments, wherein the DNA comprises cell free DNA (cfDNA).

Embodiment 24. The method of any one of embodiments 5-23, wherein the DNA comprises DNA isolated from intact cells originally present in the sample.

Embodiment 25. The method of any one of the preceding embodiments, comprising determining a ratio of levels or quantities of immune cells types based on the determined levels or quantities of the plurality of immune cell types.

Embodiment 26. The method of the immediately preceding embodiment, wherein the ratio numerator comprises the level or quantity of neutrophils, monocytes, or both neutrophils and monocytes.

Embodiment 27. The methods of embodiment 25 or 26, wherein the ratio denominator comprises the level or quantity of T cells, B cells, NK cells, or total lymphocytes.

Embodiment 28. The method of any one of embodiments 25-27, wherein the ratio numerator comprises the level or quantity of neutrophils, and the ratio denominator comprises the level or quantity of total lymphocytes.

Embodiment 29. The method of any one of embodiments 25-27, wherein the ratio numerator comprises the level or quantity of monocytes, and the ratio denominator comprises the level or quantity of T cells.

Embodiment 30. The method of any one of the preceding embodiments, comprising determining the frequency of turnover of at least one of the plurality of immune cell types.

Embodiment 31. The method of the immediately preceding embodiment, wherein the turnover comprises proliferation.

Embodiment 32. The method of embodiment 30, wherein the turnover comprises apoptosis.

Embodiment 33. The method of any one of the preceding embodiments, wherein the method comprises determining the level of at least one cell type other than an immune cell type from which the DNA originated.

Embodiment 34. The method of the immediately preceding embodiment, wherein the method comprises capturing at least one epigenetic target region set comprising sequence-independent differences in target regions in DNA originating from the cell type other than an immune cell type relative to the same target regions in DNA originating from all other cell types in the sample or subsample.

Embodiment 35. The method of embodiment 33 or 34, wherein the cell type other than an immune cell type is not a blood cell type.

Embodiment 36. The method of the immediately preceding embodiment, wherein the cell type other than an immune cell type is colorectal, lung, breast, prostate, skin, stomach, bladder, liver, ovary, pancreas, squamous, salivary gland, larynx, hypopharynx, nasal, paranasal sinus, nasopharynx, or kidney.

Embodiment 37. The method of any one of the preceding embodiments, wherein the sample is obtained from a subject.

Embodiment 38. The method of the immediately preceding embodiment, comprising determining a likelihood that the subject has cancer or precancer.

Embodiment 39. The method of the immediately preceding embodiment, comprising determining a likelihood that the subject has cancer.

Embodiment 40. The method of the immediately preceding embodiments, wherein the cancer is a cancer of an immune cell type.

Embodiment 41. The method of the immediately preceding embodiment, wherein the cancer is a lymphocytic cancer.

Embodiment 42. The method of the immediately preceding embodiments, wherein the cancer is a leukemia, a lymphoma, or a myeloma.

Embodiment 43. The method of any one of embodiments 38-40, wherein the cancer is a myeloid cancer.

Embodiment 44. The method of embodiment 38 or 39, wherein the cancer is a cancer of a cell or tissue type other than an immune cell type.

Embodiment 45. The method of any one of embodiments 38, 39, or 44, wherein the cancer or precancer is a cancer or precancer other than a hematological cancer or precancer, or wherein the cancer or precancer is a solid tumor cancer, optionally wherein the solid tumor cancer is a carcinoma or sarcoma.

Embodiment 46. The method of embodiment 44 or 45, wherein the cancer is colorectal cancer, lung cancer, breast cancer, prostate cancer, skin cancer, stomach cancer, bladder cancer, liver cancer, ovarian cancer, pancreatic cancer, head and neck cancer, or kidney cancer.

Embodiment 46.1 The method of embodiment 46, wherein the cancer is colorectal cancer.

Embodiment 46.2 The method of embodiment 46, wherein the cancer is lung cancer.

Embodiment 46.3 The method of embodiment 46, wherein the cancer is breast cancer.

Embodiment 46.4 The method of embodiment 46, wherein the cancer is prostate cancer.

Embodiment 46.5 The method of embodiment 46, wherein the cancer is skin cancer.

Embodiment 46.6 The method of embodiment 46, wherein the cancer is stomach cancer.

Embodiment 46.7 The method of embodiment 46, wherein the cancer is bladder cancer.

Embodiment 46.8 The method of embodiment 46, wherein the cancer is liver cancer.

Embodiment 46.9 The method of embodiment 46, wherein the cancer is ovarian cancer.

Embodiment 46.10 The method of embodiment 46, wherein the cancer is pancreatic cancer.

Embodiment 46.11 The method of embodiment 46, wherein the cancer is head and neck cancer.

Embodiment 46.12 The method of embodiment 46, wherein the cancer is kidney cancer.

Embodiment 47. The method of embodiment 38, comprising determining the likelihood that the subject has precancer.

Embodiment 48. The method of the immediately preceding embodiment, wherein the precancer is an adenoma.

Embodiment 49. The method of the immediately preceding embodiment, wherein the adenoma is an advanced adenoma.

Embodiment 50. The method of any one of embodiments 47-49, wherein the precancer is a colorectal precancer, lung precancer, breast precancer, prostate precancer, skin precancer, stomach precancer, bladder precancer, liver precancer, ovarian precancer, pancreatic precancer, head and neck precancer, or kidney precancer.

Embodiment 51. The method of any one of embodiments 37-50, comprising determining the likelihood that the subject has an infection.

Embodiment 52. The method of any one of embodiments 37-51, comprising determining the likelihood that the subject has transplant rejection.

Embodiment 53. The method of any one of embodiments 38-52, wherein the determining quantities of each of the plurality of immune cell types or sequencing comprises generating a plurality of sequencing reads, and wherein the method further comprises mapping the plurality of sequence reads to one or more reference sequences to generate mapped sequence reads, and processing the mapped sequence reads to determine the likelihood that the subject has cancer, precancer, infection, or transplant rejection.

Embodiment 54. The method of any one of the preceding embodiments, wherein the sample is obtained from a subject who was previously diagnosed with a cancer and received one or more previous cancer treatments, optionally wherein the sample is obtained at one or more preselected time points following the one or more previous cancer treatments.

Embodiment 55. The method of the immediately preceding embodiment, further comprising determining a cancer recurrence score, optionally wherein a cancer recurrence status of the subject is determined to be at risk for cancer recurrence when the cancer recurrence score is determined to be at or above a predetermined threshold or the cancer recurrence status of the subject is determined to be at lower risk for cancer recurrence when the cancer recurrence score is below the predetermined threshold.

Embodiment 56. The method of the immediately preceding embodiment, further comprising comparing the cancer recurrence score of the subject with a predetermined cancer recurrence threshold, wherein the subject is classified as a candidate for a subsequent cancer treatment when the cancer recurrence score is above the cancer recurrence threshold or not a candidate for a subsequent cancer treatment when the cancer recurrence score is below the cancer recurrence threshold.

Embodiment 57. The method of any one of embodiments 2, 4, 6, or 8-56, wherein the capturing comprises capturing sequence-variable target regions.

Embodiment 57.1 The method of embodiment 57, wherein the sequence-variable target regions comprise single nucleotide variations relative to a reference sequence.

Embodiment 57.2 The method of embodiment 57 or 57.1, wherein the sequence-variable target regions comprise indels relative to a reference sequence.

Embodiment 58. The method of the immediately preceding embodiment, wherein the capturing comprises contacting the DNA with target-specific probes specific for the at least one epigenetic target region set and target-specific probes specific for the sequence-variable target regions.

Embodiment 59. The method of any one of embodiments 5-58, wherein the modified cytosine is methyl cytosine.

Embodiment 60. The method of any one of embodiments 5-58, wherein the agent that recognizes a modified cytosine is a methyl binding reagent.

Embodiment 61. The method of the immediately preceding embodiment, wherein the methyl binding reagent is an antibody.

Embodiment 62. The method of embodiment 60, wherein the agent that recognizes a modified cytosine is a methyl binding protein or comprises a methyl binding domain.

Embodiment 63. The method of embodiments 60-62, wherein the methyl binding reagent specifically recognizes 5-methylcytosine.

Embodiment 64. The method of embodiments 60-63, wherein the methyl binding reagent is immobilized on a solid support.

Embodiment 65. The method of any one of embodiments 5-64, wherein the partitioning comprises immunoprecipitation of methylated DNA.

Embodiment 66. The method of any one of embodiments 5-58, wherein the partitioning comprises partitioning on the basis of binding to a protein, optionally wherein the protein is a methylated protein, an acetylated protein, an unmethylated protein, an unacetylated protein; and/or optionally wherein the protein is a histone.

Embodiment 67. The method of the immediately preceding embodiment, wherein the partitioning comprises contacting the DNA of the sample with a binding reagent which is specific for the protein and is immobilized on a solid support.

Embodiment 68. The method of any one of embodiments 5-67, comprising contacting at least one subsample with a restriction enzyme prior to the capturing or sequencing, optionally wherein the contacting occurs after partitioning the sample into the plurality of subsamples.

Embodiment 69. The method of the immediately preceding embodiment, wherein the restriction enzyme is a MDRE.

Embodiment 70. The method of the immediately preceding embodiment, wherein the second subsample is contacted with the MDRE.

Embodiment 71. The method of any one of embodiments 68-70, wherein the restriction enzyme is a MSRE.

Embodiment 72. The method of the immediately preceding embodiment, wherein the first subsample is contacted with the MSRE.

Embodiment 73. The method of any one of the preceding embodiments, wherein the method comprises ligating adapters to the DNA, thereby producing adapter-ligated DNA.

Embodiment 74. The method of the immediately preceding embodiment, wherein the adapter-ligated DNA is amplified prior to the sequencing.

Embodiment 75. The method of any one of embodiments 5-74, wherein the subsamples are pooled prior to the sequencing.

Embodiment 76. The method of any one of embodiments 1-4 or 6-75, wherein the epigenetic target region set comprises hypomethylation variable target regions comprising DNA sequences that are differentially hypomethylated in a plurality of immune cell types.

Embodiment 77. The method of any one of embodiments 3, 4, 7, or 10-76, wherein the hypomethylation variable target regions comprise DNA sequences that are differentially hypomethylated in a plurality of immune cell types.

Embodiment 78. The method of any one of embodiments 3, 4, 76, or 77, wherein the DNA sequences that are differentially hypomethylated in a plurality of immune cell types comprise a detectably lower degree of methylation in at least one immune cell type than the degree of methylation of the same sequence in any other immune cell type in the sample.

Embodiment 79. The method of any one of embodiments 3, 4, 76, or 77, wherein the DNA sequences that are differentially hypomethylated in a plurality of immune cell types comprise a detectably lower degree of methylation in at least one immune cell type than the degree of methylation of the same sequence in any other immune cell type.

Embodiment 80. The method of any one of embodiments 3, 4, 76, or 77, wherein the DNA sequences that are differentially hypomethylated in a plurality of immune cell types comprise a detectably lower degree of methylation in one immune cell type than the degree of methylation of the same sequence in any other immune cell type in the sample.

Embodiment 81. The method of any one of embodiments 3, 4, 76, or 77, wherein the DNA sequences that are differentially hypomethylated in a plurality of immune cell types comprise a detectably lower degree of methylation in one immune cell type than the degree of methylation of the same sequence in any other immune cell type.

Embodiment 82. The method of any one of embodiments 3, 4, 76, or 77, wherein the DNA sequences that are differentially hypomethylated in a plurality of immune cell types comprise a detectably lower degree of methylation in at least one immune cell type than the degree of methylation of the same sequence in at least one other immune cell type and detectably lower than the degree of methylation of the same sequence in at least one non-immune cell type in the sample.

Embodiment 83. The method of any one of embodiments 3, 4, 76, or 77, wherein the DNA sequences that are differentially hypomethylated in a plurality of immune cell types comprise a detectably lower degree of methylation in at least one immune cell type than the degree of methylation of the same sequence in at least one other immune cell type and detectably lower than the degree of methylation of the same sequence in any non-immune cell type in the sample.

Embodiment 83.1 The method of any one of embodiments 3, 4, 7, or 10-77, wherein the differentially hypomethylated DNA comprises a decreased level or degree of methylation in one cell type relative to DNA comprising the same genetic information in one or more other cell types.

Embodiment 83.2 The method of any one of embodiments 3, 4, 7, or 10-77, wherein the differentially hypomethylated DNA comprises a decreased level or degree of methylation in one or more immune cell types relative to DNA comprising the same genetic information in all other immune cell types.

Embodiment 83.3 The method of any one of embodiments 3, 4, 7, or 10-77, wherein the differentially hypomethylated DNA comprises a decreased level or degree of methylation in one or more immune cell types relative to DNA comprising the same genetic information in all other blood cell types, optionally wherein the level or degree of methylation in DNA of all other blood cell types is expressed as a weighted average according to abundance of the DNA from the cell types.

Embodiment 84. The method of any one of embodiments 78-83, wherein the detectably lower degree of methylation is one fewer methylated cytosine than the same sequence in the other cell types.

Embodiment 85. The method of any one of embodiments 78-83, wherein the detectably lower degree of methylation is two fewer methylated cytosines than the same sequence in the other cell types.

Embodiment 86. The method of any one of embodiments 78-83, wherein the detectably lower degree of methylation is three fewer methylated cytosines than the same sequence in the other cell types.

Embodiment 87. The method of any one of embodiments 78-83, wherein the detectably lower degree of methylation is four fewer methylated cytosines than the same sequence in the other cell types.

Embodiment 88. The method of any one of embodiments 78-83, wherein the detectably lower degree of methylation is five or more fewer methylated cytosines than the same sequence in the other cell types.

Embodiment 89. The method of any one of embodiments 1-4 or 6-88, wherein the epigenetic target region set comprises hypermethylation variable target regions comprising DNA sequences that are differentially hypermethylated in a plurality of immune cell types.

Embodiment 90. The method of embodiment 89, wherein the DNA sequences that are differentially hypermethylated in a plurality of immune cell types comprise a detectably higher degree of methylation in at least one immune cell type than the degree of methylation of the same sequence in any other immune cell type in the sample.

Embodiment 91. The method of embodiment 89, wherein the DNA sequences that are differentially hypermethylated in a plurality of immune cell types comprise a detectably higher degree of methylation in at least one immune cell type than the degree of methylation of the same sequence in any other immune cell type.

Embodiment 92. The method of embodiment 89, wherein the DNA sequences that are differentially hypermethylated in a plurality of immune cell types comprise a detectably higher degree of methylation in one immune cell type than the degree of methylation of the same sequence in any other immune cell type in the sample.

Embodiment 93. The method of embodiment 89, wherein the DNA sequences that are differentially hypermethylated in a plurality of immune cell types comprise a detectably higher degree of methylation in one immune cell type than the degree of methylation of the same sequence in any other immune cell type.

Embodiment 94. The method of embodiment 89, wherein the DNA sequences that are differentially hypermethylated in a plurality of immune cell types comprise a detectably higher degree of methylation in at least one immune cell type than the degree of methylation of the same sequence in at least one other immune cell type and detectably lower than the degree of methylation of the same sequence in at least one non-immune cell type in the sample.

Embodiment 95. The method of embodiment 89, wherein the DNA sequences that are differentially hypermethylated in a plurality of immune cell types comprise a detectably higher degree of methylation in at least one immune cell type than the degree of methylation of the same sequence in at least one other immune cell type and detectably lower than the degree of methylation of the same sequence in any non-immune cell type in the sample.

Embodiment 95.1 The method of embodiment 89, wherein the differentially hypermethylated DNA comprises an increased level or degree of methylation in one cell type relative to DNA comprising the same genetic information in one or more other cell types.

Embodiment 95.2 The method of embodiment 89, wherein the differentially hypermethylated DNA comprises an increased level or degree of methylation in one or more immune cell types relative to DNA comprising the same genetic information in all other immune cell types.

Embodiment 95.3 The method of embodiment 89, wherein the differentially hypermethylated DNA comprises an increased level or degree of methylation in one or more immune cell types relative to DNA comprising the same genetic information in all other blood cell types, optionally wherein the level or degree of methylation in DNA of all other blood cell types is expressed as a weighted average according to abundance of the DNA from the cell types.

Embodiment 96. The method of any one of embodiments 90-95 wherein the detectably higher degree of methylation is one more cytosine methylation than the same sequence in the other cell types.

Embodiment 97. The method of any one of embodiments 90-95, wherein the detectably higher degree of methylation is two more cytosine methylations than the same sequence in the other cell types.

Embodiment 98. The method of any one of embodiments 90-95, wherein the detectably higher degree of methylation is three more cytosine methylations than the same sequence in the other cell types.

Embodiment 99. The method of any one of embodiments 90-95, wherein the detectably higher degree of methylation is four more cytosine methylations than the same sequence in the other cell types.

Embodiment 100. The method of any one of embodiments 90-95, wherein the detectably higher degree of methylation is five more or more than five more cytosine methylations than the same sequence in the other cell types.

Embodiment 101. The method of any one of the preceding embodiments, comprising determining the quantity or detecting the level of DNA in the sample originating from erythrocytes or erythrocyte progenitors.

Embodiment 102. The method of any one of the preceding embodiments, comprising determining the quantity or detecting the level of DNA in the sample originating from granulocytes.

Embodiment 103. The method of any one of embodiments 3, 4, or 76-102, wherein the detectably lower or higher degree of methylation is present in samples from at least one group of donors relative to another group of donors, optionally wherein the at least one group of donors have a cancer that responds to a therapy and the another group of donors have a cancer that does not respond to the therapy.

### BRIEF DESCRIPTION OF THE DRAWINGS

FIG. 1A shows an exemplary workflow according to certain embodiments disclosed herein.

FIG. 1B is a heat map showing methylation level of loci identified as having cell type-specific or cell cluster-specific differential methylation as a function of cell type or cell cluster. DMS and DMR indicate differentially methylated sites (e.g., individual CpGs) and differentially methylated regions (comprising a plurality of DMSs).

FIG. 1C shows relative methylation of exemplary hypermethylated regions identified for the indicated cell types and cell clusters in samples from individuals with colorectal cancer (CRC) and samples from cancer-free individuals as boxplots. The boxplots show the distribution of methylation scores in CRC and cancer-free samples. The methylation score is the total molecules in the methylated fractions (hypermethylated and intermediate partitions) normalized by the input cfDNA. In each box in the boxplots, the rightmost line, the middle line and the leftmost line correspond to the 75% quantile (Q75), 50% quantile or median (Q50) and 25% quantile (Q25) of the methylation scores across samples. The rightmost whisker and the leftmost whisker correspond to Q75+1.5*interquartile range (IQR) and Q25−1.5 IQR, where IQR is Q75-Q25. The dots are outliers whose values are beyond the range marked by the whiskers.

FIG. 1D shows relative methylation of exemplary hypermethylated regions identified for the indicated cell types and cell clusters in samples from individuals with the indicated types of early-stage cancer and samples from cancer-free individuals as boxplots. The boxplots show the distribution of methylation scores in the samples. The methylation score is the total molecules in the methylated fraction (hypermethylated and intermediate partitions) normalized by the input cfDNA. In each box in the boxplots, the rightmost line, the middle line and the leftmost line correspond to the 75% quantile (Q75), 50% quantile or median (Q50) and 25% quantile (Q25) of the methylation scores across samples. The rightmost whisker and the leftmost whisker correspond to Q75+1.5*interquartile range (IQR) and Q25−1.5 IQR, where IQR is Q75-Q25. The dots are outliers whose values are beyond the range marked by the whiskers.

FIG. 1E shows relative methylation of exemplary hypermethylated regions identified for the indicated cell types and cell clusters in samples from individuals with the indicated types of late-stage cancer and samples from cancer-free individuals as boxplots. The boxplots show the distribution of methylation scores in the samples. The methylation score is the total molecules in the methylated fraction (hypermethylated and intermediate partitions) normalized by the input cfDNA. In each box in the boxplots, the rightmost line, the middle line and the leftmost line correspond to the 75% quantile (Q75), 50% quantile or median (Q50) and 25% quantile (Q25) of the methylation scores across samples. The rightmost whisker and the leftmost whisker correspond to Q75+1.5*interquartile range (IQR) and Q25−1.5 IQR, where IQR is Q75-Q25. The dots are outliers whose values are beyond the range marked by the whiskers.

FIG. 2 is a schematic diagram of an example of a system suitable for use with some embodiments of the disclosure.

FIG. 3 shows estimated percentages of B cells, granulocytes, NK cells, T cells, and erythrocyte progenitors as contributors to cfDNA in blood samples from cancer free subjects and subjects having colorectal cancer (crc).

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### Definitions

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments.

As used herein, “partitioning” of nucleic acids, such as DNA molecules, means separating, fractionating, sorting, or enriching a sample or population of nucleic acids into a plurality of subsamples or subpopulations of nucleic acids based on one or more modifications or features that is in different proportions in each of the plurality of subsamples or subpopulations. Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, a modification or other feature is present in “a greater proportion” in a first sample or population of nucleic acid than in a second sample or population when the fraction of nucleotides with the modification or other feature is higher in the first sample or population than in the second population. For example, if in a first sample, one tenth of the nucleotides are mC, and in a second sample, one twentieth of the nucleotides are mC, then the first sample comprises the cytosine modification of 5-methylation in a greater proportion than the second sample.

As used herein, the form of the “originally isolated” sample refers to the composition or chemical structure of a sample at the time it was isolated and before undergoing any procedure that changes the chemical structure of the isolated sample. Similarly, a feature that is “originally present” in DNA molecules refers to a feature present in “original DNA molecules” or in DNA molecules “originally comprising” the feature before the DNA molecules undergo a procedure that changes the chemical structure of DNA molecules.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

“Capturing” one or more target nucleic acids or one or more nucleic acids comprising at least one target region refers to preferentially isolating or separating the one or more target nucleic acids or one or more nucleic acids comprising at least one target region from non-target nucleic acids or from nucleic acids that do not comprise at least one target region.

A “captured set” of nucleic acids or “captured” nucleic acids refers to nucleic acids that have undergone capture.

As used herein, a “capture moiety” is a molecule that allows affinity separation of molecules, such as nucleic acids, linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “cell cluster” or “cluster” is a plurality of related cell types, e.g., immune cell types. In some embodiments, the cell types within a cluster have similar DNA methylation profiles, e.g., in a plurality of hypermethylation variable target regions and/or hypomethylation variable target regions.

A “target region” refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity).

“Specifically binds” in the context of a primer, a probe, or other oligonucleotide and a target sequence means that under appropriate hybridization conditions, the primer, oligonucleotide, or probe hybridizes to its target sequence, or replicates thereof, to form a stable hybrid, while at the same time formation of stable non-target hybrids is minimized. Thus, a primer or probe hybridizes to a target sequence or replicate thereof to a sufficiently greater extent than to a non-target sequence, to ultimately enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, N Y, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

“Sequence-variable target regions” refer to target regions that may exhibit changes in sequence such as nucleotide substitutions (i.e., single nucleotide variations), insertions, deletions, or gene fusions or transpositions in neoplastic cells (e.g., tumor cells and cancer cells) relative to normal cells. A sequence-variable target region set is a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, 2, or 1 nucleotides.

“Epigenetic target regions” refers to target regions that may show sequence-independent differences in different cell or tissue types (e.g., different types of immune cells) or in neoplastic cells (e.g., tumor cells and cancer cells) relative to normal cells; or that may show sequence-independent differences (i.e., in which there is no change to the nucleotide sequence, e.g., differences in methylation, nucleosome distribution, or other epigenetic features) in DNA, such as cfDNA, from different cell types or from subjects having cancer relative to DNA, such as cfDNA, from healthy subjects, or in cfDNA originating from different cell or tissue types that ordinarily do not substantially contribute to cfDNA (e.g., immune, lung, colon, etc.) relative to background cfDNA (e.g., cfDNA that originated from hematopoietic cells). Examples of sequence-independent changes include, but are not limited to, changes in methylation (increases or decreases), nucleosome distribution, cfDNA fragmentation patterns, CCCTC-binding factor (“CTCF”) binding, transcription start sites (e.g., with respect to any one of more of binding of RNA polymerase components, binding of regulatory proteins, fragmentation characteristics, and nucleosomal distribution), and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions. An epigenetic target region set is a set of epigenetic target regions.

As used herein, a “differentially methylated region” refers to a region of DNA having a detectably different degree of methylation in at least one cell or tissue type relative to the degree of methylation in the same region of DNA from at least one other cell or tissue type; or having a detectably different degree of methylation in at least one cell or tissue type obtained from a subject having a disease or disorder relative to the degree of methylation in the same region of DNA in the same cell or tissue type obtained from a healthy subject. In some embodiments, a differentially methylated region has a detectably higher degree of methylation (e.g., a hypermethylated region) in at least one cell or tissue type, such as at least one immune cell type, relative to the degree of methylation in the same region of DNA from at least one other cell or tissue type, such as other immune cell types and/or cell types that contribute to cfDNA in healthy individuals, or from the same cell or tissue type from a healthy subject. In some embodiments, a differentially methylated region has a detectably lower degree of methylation (e.g., a hypomethylated region) in at least one cell or tissue type, such as at least one immune cell type, relative to the degree of methylation in the same region of DNA from at least one other cell or tissue type, such as other immune cell types and/or cell types that contribute to cfDNA in healthy individuals, or from the same cell or tissue type from a healthy subject.

A nucleic acid is “produced by a tumor” or is “circulating tumor DNA” (“ctDNA”) if it originated from a tumor cell. Tumor cells are neoplastic cells that originated from a tumor, regardless of whether they remain in the tumor or become separated from the tumor (as in the cases, e.g., of metastatic cancer cells and circulating tumor cells). As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The term “methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′->3′ direction of the nucleic acid sequence). In some embodiments, DNA methylation refers to addition of a methyl group to adenine, such as in N6-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer

The term “hypermethylation” refers to an increased level or degree of methylation of nucleic acid molecule(s) relative to the other nucleic acid molecules comprising the same genetic information within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules comprising the same genetic information within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded. Suitable agents include agents that recognize modified nucleotides in double-stranded DNA, single-stranded DNA, and both double-stranded and single-stranded DNA.

The terms “or a combination thereof” and “or combinations thereof” as used herein refers to any and all permutations and combinations of the listed terms preceding the term. For example, “A, B, C, or combinations thereof is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### Exemplary Methods

a. Immune Cell Type Identification and Quantification

In some embodiments, methods disclosed herein comprise sequencing cfDNA from a sample and determining methylation levels for a plurality of target regions comprising DNA sequences that are differentially methylated in a plurality of immune cell types. In some embodiments, methods disclosed herein comprise capturing at least an epigenetic target region set from cfDNA or a subsample thereof, comprising contacting the cfDNA or subsample thereof with target-specific probes specific for the at least one epigenetic target region set, wherein the epigenetic target region set comprises target regions comprising DNA sequences that are differentially methylated in a plurality of immune cell types, and determining methylation levels for the target regions. In some embodiments, methods disclosed herein comprise sequencing cfDNA and determining methylation levels for a plurality of hypomethylation variable target regions comprising DNA sequences that are differentially hypomethylated in a plurality of immune cell types. Target regions that are differentially hypomethylated in a plurality of immune cell types show a lower level of methylation in the plurality of immune cell types than in other cell types. In some embodiments, methods disclosed herein comprise capturing at least an epigenetic target region set from cfDNA or a subsample thereof, comprising contacting the cfDNA or subsample thereof with target-specific probes specific for the at least one epigenetic target region set, wherein the epigenetic target region set comprises hypomethylation variable target regions comprising DNA sequences that are differentially hypomethylated in a plurality of immune cell types, and determining methylation levels for the target regions. In any of these embodiments, methylation levels can be determined using partitioning, methylation-sensitive conversion such as bisulfite conversion, direct detection during sequencing, or any other suitable approach. Various approaches are described herein.

The methylation levels can be used to determine quantities of each of a plurality of immune cell types from which the cfDNA originated. This can be useful, e.g., to detect the presence of cancer or precancer, or other conditions (e.g., infection, transplant rejection), in that the state of the immune system as reflected in the distribution of cell types that contribute to cfDNA can change as a result of such conditions. In some embodiments, the cfDNA originated from a tumor cell, and the cancer is a hematological cancer. In some embodiments, the cfDNA did not originate from a tumor cell. In some such embodiments, the cancer is not a hematological cancer. In some such embodiments, the cancer is a solid tumor cancer, e.g., a carcinoma or sarcoma. Without wishing to be bound by theory, cancers, including solid tumor cancers such as carcinomas and sarcomas, may cause changes to immune cell type distribution, including with respect to differentiated immune cell types and immune cell activation states, relative to the immune cell distribution in a healthy subject or subject that does not have cancer. Such changes may be detected in the methods herein and can be useful in detecting cancer as well as determining cancer prognosis and/or treatment options.

In some embodiments, methods disclosed herein comprise steps of partitioning a sample comprising DNA by contacting the DNA with an agent that recognizes a modified cytosine in the DNA, sequencing the DNA, and determining levels of each of a plurality of immune cell types from which the DNA originated. The levels of immune cell types may be expressed, e.g., as relative amounts or percentages for each cell type being quantified. Such determination is illustrated, e.g., in Example 3. In some embodiments, the methods comprise capturing or enriching an epigenetic target region set of DNA from one or more partitioned subsamples prior to sequencing. In some embodiments, the modified cytosine is methyl cytosine.

The methods herein thus allow for detection and/or identification of immune-specific differentially methylated genome regions that can be used to identify and quantify different immune cell types from which DNA in a sample originated. The immune cell types may comprise immune cells of different differentiation types, different activation types, or both different differentiation and different activation types. Indeed, differentiation status and activation status significantly overlap and often change together in a given immune cell. For example, activation of an immune cell may induce differentiation of the cell. Immune cells of different activation types include activated cells, such as cells activated by inflammatory cytokines or antigens, and suppressed cells, such as cells suppressed by Tregs. The immune cell types include activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. DNA from such cell types may be rare in samples, such as cfDNA samples, from healthy individuals, but more common in samples, such as cfDNA samples, from individuals with a disease or disorder such as cancer or a precancerous condition. In some embodiments, to distinguish DNA from closely related cell types, such as naïve and activated B cells, naïve and activated T cells, or different stages myeloid lineages, both hypermethylated regions and hypomethylated regions may be detected. In some embodiments, at least some of the differentially methylated regions are exclusively hypermethylated or exclusively hypomethylated in only one cell type or in only one cell type within a cluster. In some embodiments, at least 2, at least 3, at least 4, at least 5, at least 6, at least 7, at least 8, at least 9, or at least 10 differentially methylated regions are exclusively hypermethylated or exclusively hypomethylated in only one cell type that is being identified or quantified within a cluster.

In some embodiments, determining the levels of different immune cell types from which DNA in a sample originated facilitates disease diagnosis or identification of appropriate treatments. In some embodiments, a change in the levels of one or more immune cell types is indicative of the presence of a disease or disorder in a subject, such as cancer, precancer, an infection, transplant rejection, or other disorder that causes changes in the relative amounts of certain immune cell types relative to the amounts present in a healthy subject. In some embodiments, changes in both the levels of one or more immune cell types in combination with sequence-independent changes in epigenetic target regions are indicative of the presence of a disease or disorder in a subject, such as cancer, precancer, an infection, transplant rejection, or other disorder that causes changes in the relative amounts of certain immune cell types and epigenetic changes relative to a healthy subject. In some embodiments, the methods facilitate identification of appropriate treatments based on the likelihood that a subject will respond to the treatment. In some such embodiments, determining the levels of DNA from one or more immune cell types in a sample from a subject having a certain cancer type facilitates prediction of the clinical outcome for immunotherapy in the subject. Where determining the levels of different immune cell types facilitates disease diagnosis, identification of appropriate treatments and association with therapeutic response, the thresholds for disease diagnosis and for identification of appropriate treatments may be the same or different. The levels can be determined based on a count of molecules corresponding to different immune cell types, or the relative frequency of such molecules or any value or ratio based on a count of molecules corresponding to one or more different immune cell types.

b. Partitioning the Sample into a Plurality of Subsamples

In some embodiments described herein, different forms of DNA (e.g., hypermethylated and hypomethylated DNA) are physically partitioned based on one or more characteristics of the DNA. This approach can be used to determine, for example, whether certain sites or regions are hypermethylated or hypomethylated. Partitioning can be performed before attaching adapters to DNA molecules in the sample, e.g., so as to facilitate including partition tags in the adapters. Partition tags can be used to identify which partition a molecule was found in. Following partitioning (and attachment of adapters if applicable), further steps such as amplification, target capture, and sequencing may be performed.

Methylation profiling can involve determining methylation patterns across different regions of the genome. For example, after partitioning molecules based on extent of methylation (e.g., relative number of methylated nucleobases per molecule) and further steps as discussed above including sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of particular immune cell types, such as rare immune cell types, tumor cells or cells of a type that does not normally contribute to the DNA sample being analyzed (such as cfDNA).

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

In some embodiments, partitioning is on the basis of one or more characteristics such as methylation. Molecules can be sorted according to other characteristics, such as sequence length, nucleosome binding, sequence mismatch, immunoprecipitation, and/or proteins that bind to DNA, using appropriate techniques as part of data analysis or partitioning as applicable. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by the procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

The binding of partitioning agents to particular nucleic acids and the partitioning of the nucleic acids into subsamples may occur to a certain extent or may occur in an essentially binary manner. In some instances, nucleic acids comprising a greater proportion of a certain modification bind to the agent at a greater extent than nucleic acids comprising a lesser proportion of the modification. Similarly, the partitioning may produce subsamples comprising greater and lesser proportions of nucleic acids comprising a certain modification. Alternatively, the partitioning may produce subsamples comprising essentially all or none of the nucleic acids comprising the modification. In all instances, various levels of modifications may be sequentially eluted from the partitioning agent.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner® Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning of the sample into a plurality of subsamples is performed by contacting the nucleic acids with an antibody that recognizes a modified nucleobase in the DNA, which may be is a modified cytosine or a product of the procedure that affects the first nucleobase in the DNA differently from the second nucleobase in the DNA of the sample. In some embodiments, the modified nucleobase is 5mC. In some embodiments, the modified nucleobase is 5caC. In some embodiments, the modified nucleobase is dihydrouracil (DHU). In some embodiments, the antibody that recognizes a modified nucleobase in the DNA is used to partition single-stranded DNA.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, an MBD binds to 5mC and 5hmC. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, sample DNA (e.g., between 5 and 200 ng) is mixed with methyl binding domain (MBD) buffer and magnetic beads conjugated with MBD proteins and incubated overnight. Methylated DNA (hypermethylated DNA) binds the MBD protein on the magnetic beads during this incubation. Non-methylated (hypomethylated DNA) or less methylated DNA (intermediately methylated) is washed away from the beads with buffers containing increasing concentrations of salt. For example, one, two, or more fractions containing non-methylated, hypomethylated, and/or intermediately methylated DNA may be obtained from such washes. Finally, a high salt buffer is used to elute the heavily methylated DNA (hypermethylated DNA) from the MBD protein. In some embodiments, these washes result in three partitions (hypomethylated partition, intermediately methylated fraction and hypermethylated partition) of DNA having increasing levels of methylation.

In some embodiments, partitioning procedures may result in imperfect sorting of DNA molecules among the subsamples. For example, a minority of the molecules in an unmethylated or hypomethylated subsample may be highly modified (e.g., hypermethylated), and/or a minority of the molecules in a hypermethylated subsample may be unmodified or mostly unmodified (e.g., unmethylated or mostly unmethylated). Such molecules are considered nonspecifically partitioned.

In some embodiments, nonspecifically partitioned molecules are removed using a methylation-dependent nuclease, e.g., a methylation dependent restriction enzyme (MDRE), digesting/cleaving the DNA where the restriction enzyme (RE) recognition site contains a methylated nucleotide but not cleaving the DNA where the restriction enzyme (RE) recognition site contains an unmethylated nucleotide. In some embodiments, nonspecifically partitioned molecules are removed using a methylation sensitive nuclease, e.g., a methylation sensitive restriction enzyme (MSRE), digesting/cleaving the DNA where the restriction enzyme (RE) recognition site contains an unmethylated nucleotide but not cleaving the DNA where the restriction enzyme (RE) recognition site contains a methylated nucleotide. For example, in some embodiments, a hypomethylated subsample is contacted with a methylation-dependent nuclease, such as a methylation-dependent restriction enzyme, thereby degrading nonspecifically partitioned DNA, e.g., methylated DNA, in the subsample. Alternatively, or in addition, a hypermethylated subsample is contacted with a methylation-sensitive endonuclease, such as a methylation-sensitive restriction enzyme, thereby degrading nonspecifically partitioned DNA in the subsample.

Degradation of nonspecifically partitioned DNA in one or more partitioned subsamples may improve the performance of methods that rely on accurate partitioning of DNA on the basis of a cytosine modification. For example, such degradation may provide improved sensitivity and/or simplify downstream analyses. In some embodiments, partitioning DNA on the basis of a modification, such as methylation, then removing nonspecifically partitioned DNA using MDREs and/or MSREs as described herein provides improved efficiency and/or cost over DNA analysis methods comprising procedures that affect a first nucleobase differently from a second nucleobase, such as bisulfite sequencing or bisulfite conversion.

In some embodiments, one or more nucleases are used to degrade nonspecifically partitioned DNA molecules. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting a subsample with more than one methylation-dependent restriction enzyme can more completely degrade nonspecifically partitioned hypermethylated DNA. Contacting a subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used.

In some embodiments, a methylation-sensitive nuclease comprises one or more of AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp1406I, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I.

In some embodiments, the partitions of DNA are desalted and concentrated in preparation for enzymatic steps of library preparation.

c. Adapter Ligation or Addition

In some embodiments, adapters are added to the DNA. This may be done concurrently with an amplification procedure, e.g., by providing the adapters in a 5′ portion of a primer (where PCR is used, this can be referred to as library prep-PCR or LP-PCR). In some embodiments, adapters are added by other approaches, such as ligation. In some such methods, prior to partitioning or prior to capturing, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA is partitioned, comprising contacting the DNA with an agent that preferentially binds to nucleic acids bearing an epigenetic modification. The nucleic acids are partitioned into at least two subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

d. Tagging

“Tagging” DNA molecules is a procedure in which a tag is attached to or associated with the DNA molecules. Tags can be molecules, such as nucleic acids, containing information that indicates a feature of the molecule with which the tag is associated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. Tags can be used to label the individual polynucleotide population partitions so as to correlate the tag (or tags) with a specific partition. Alternatively, tags can be used in embodiments that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In some embodiments, the partition tags can serve as identifiers of the source partition and the molecule, i.e., different partitions are tagged with different sets of molecular tags, e.g., comprised of a pair of barcodes. In this way, the one or more molecular barcodes attached to the molecule indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

e. Enriching/Capturing Step; Amplification

Methods disclosed herein can comprise capturing DNA, such as cfDNA target regions. In some embodiments, the capturing comprises contacting the DNA with probes (e.g., oligonucleotides) specific for the target regions. Enrichment or capture may be performed on any sample or subsample described herein using any suitable approach known in the art.

In some embodiments, enrichment or capture is performed after attachment of adapters to sample molecules. In some embodiments, enrichment or capture is performed after a partitioning step. In some embodiments, enrichment or capture is performed after an amplification step. In some embodiments, sample molecules are partitioned, then adapters are attached, then sample molecules are amplified, and then the amplified molecules are subjected to enrichment or capture. The enriched or captured molecules may then be subjected to another amplification and then sequenced.

In some embodiments, the probes specific for the target regions comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, the methods herein comprise enriching for or capturing DNA comprising epigenetic and/or sequence-variable target regions. Such regions may be captured from an aliquot of a sample (e.g., a sample that has undergone attachment of adapters and amplification), while the step of partitioning the DNA with an agent that recognizes a modified cytosine, such as methyl cytosine, is performed on a separate aliquot of the sample. Enriching for or capturing DNA comprising epigenetic and/or sequence-variable target regions may comprise contacting the DNA with a first or second set of target-specific probes. Such target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from the first subsample or the second subsample, e.g., the first subsample and the second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, methods described herein comprise capturing a plurality of sets of target regions of cfDNA obtained from a subject. The target regions may comprise differences depending on whether they originated from a tumor or from healthy cells or from a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In some embodiments, the DNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured DNA, e.g., to different degrees of sequencing depth for the epigenetic and sequence-variable target region sets, consistent with the discussion herein.

In some embodiments, a capturing step is performed with probes for a sequence-variable target region set and probes for an epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, a capturing step is performed with a sequence-variable target region probe set in a first vessel and with an epigenetic target region probe set in a second vessel, or a contacting step is performed with a sequence-variable target region probe set at a first time and a first vessel and an epigenetic target region probe set at a second time before or after the first time. This approach allows for preparation of separate first and second compositions comprising captured DNA corresponding to a sequence-variable target region set and captured DNA corresponding to an epigenetic target region set. The compositions can be processed separately as desired (e.g., to partition based on methylation as described herein) and pooled in appropriate proportions to provide material for further processing and analysis such as sequencing.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

f. Procedures that Affect a First Nucleobase in the DNA Differently from a Second Nucleobase in the DNA

In some embodiments, methods disclosed herein comprise a step of subjecting DNA to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases comprises mC and the other comprises hmC.

In some embodiments, the procedure that affects a first nucleobase in the DNA differently from a second nucleobase in the DNA comprises bisulfite conversion. Treatment with bisulfite converts unmodified cytosine and certain modified cytosines (e.g. 5-formyl cytosine (fC) or 5-carboxylcytosine (caC)) to uracil whereas other modified cytosines (e.g., 5-methylcytosine, 5-hydroxylmethylcystosine) are not converted. Performing bisulfite conversion can facilitate identifying positions containing mC or hmC using the sequence reads. For an exemplary description of bisulfite conversion, see, e.g., Moss et al., Nat Commun. 2018; 9:5068.

In some embodiments, the procedure that affects a first nucleobase in the DNA differently from a second nucleobase in the DNA comprises oxidative bisulfite (Ox-BS) conversion. Performing Ox-BS conversion can facilitate identifying positions containing mC using the sequence reads. For an exemplary description of oxidative bisulfite conversion, see, e.g., Booth et al., Science 2012; 336:934-937.

In some embodiments, the procedure that affects a first nucleobase in the DNA differently from a second nucleobase in the DNA comprises Tet-assisted bisulfite (TAB) conversion. For example, as described in Yu et al., Cell 2012; 149:1368-80, β-glucosyl transferase can be used to protect hmC (forming 5-glucosylhydroxymethylcytosine (ghmC)), then a TET protein such as mTet1 can be used to convert mC to caC, and then bisulfite treatment can be used to convert C and caC to U while ghmC remains unaffected. Thus, when TAB conversion is used, the first nucleobase comprises one or more of unmodified cytosine, fC, caC, mC, or other cytosine forms affected by bisulfite, and the second nucleobase comprises hmC. Performing TAB conversion can facilitate identifying positions containing hmC using the sequence reads.

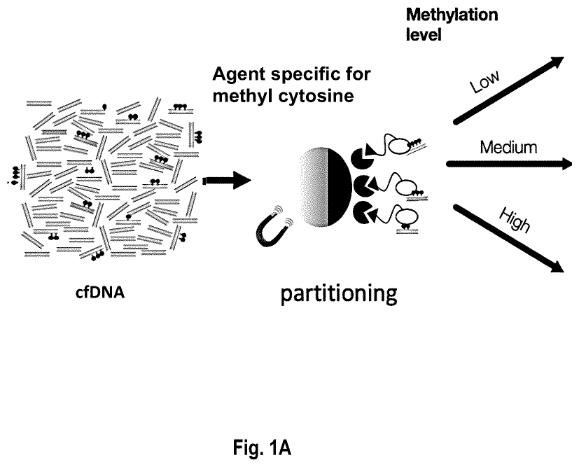

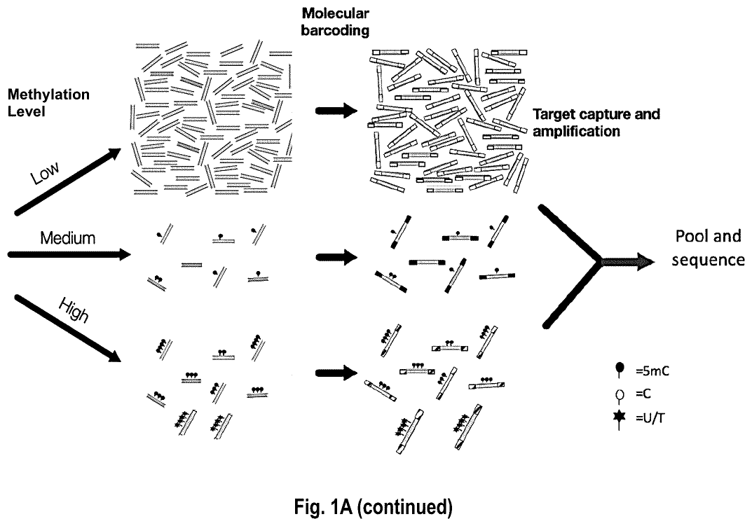

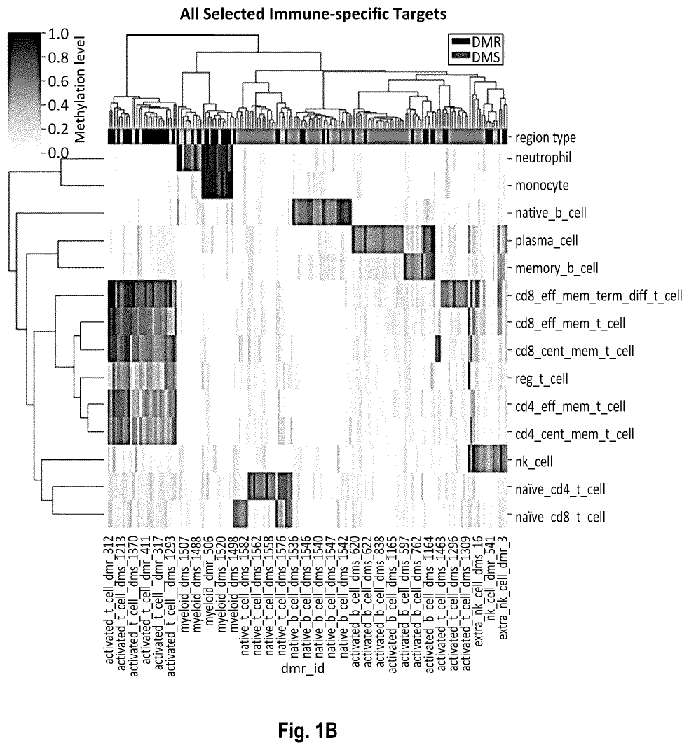

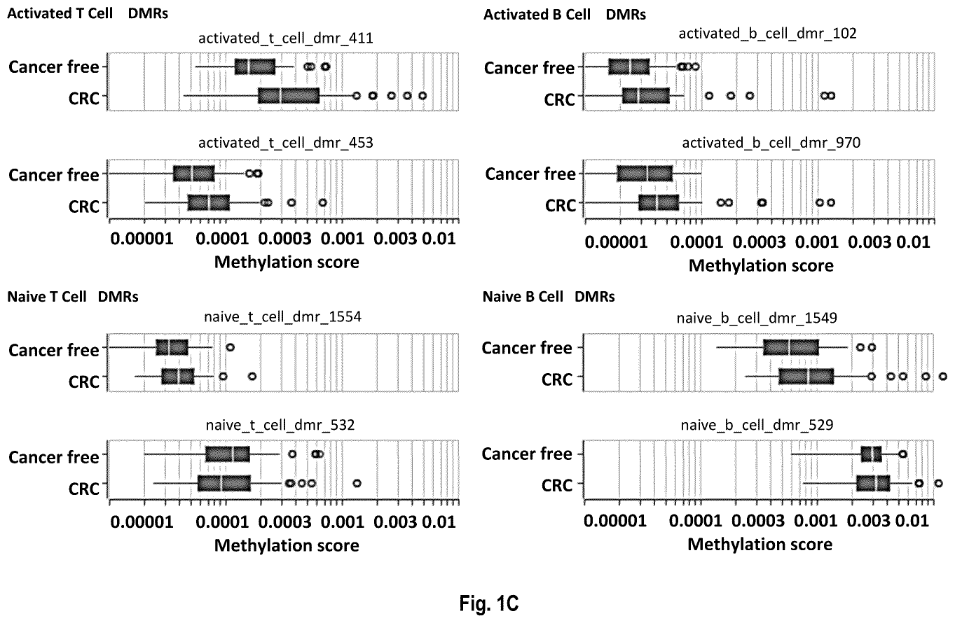

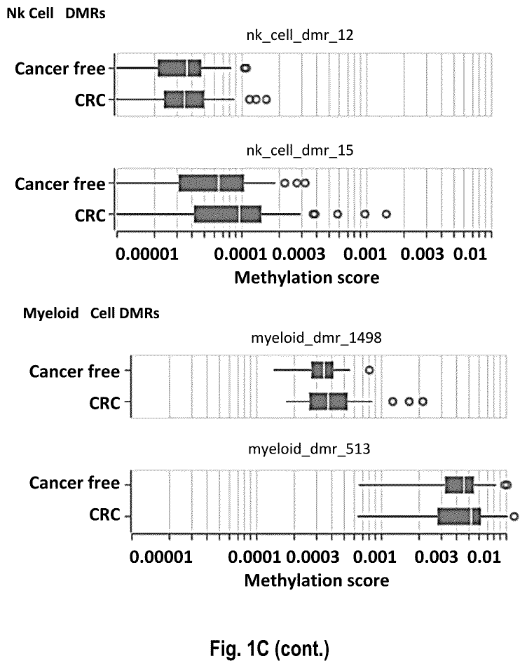

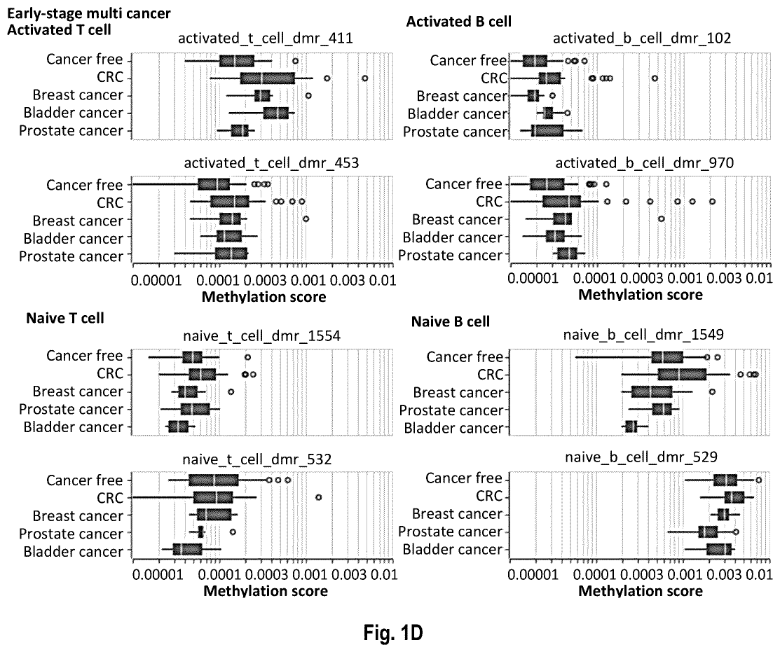

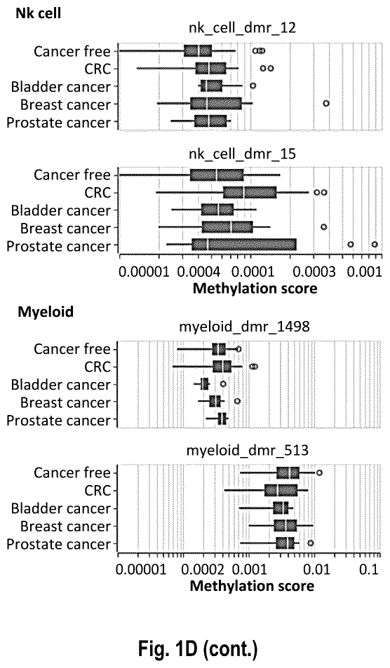

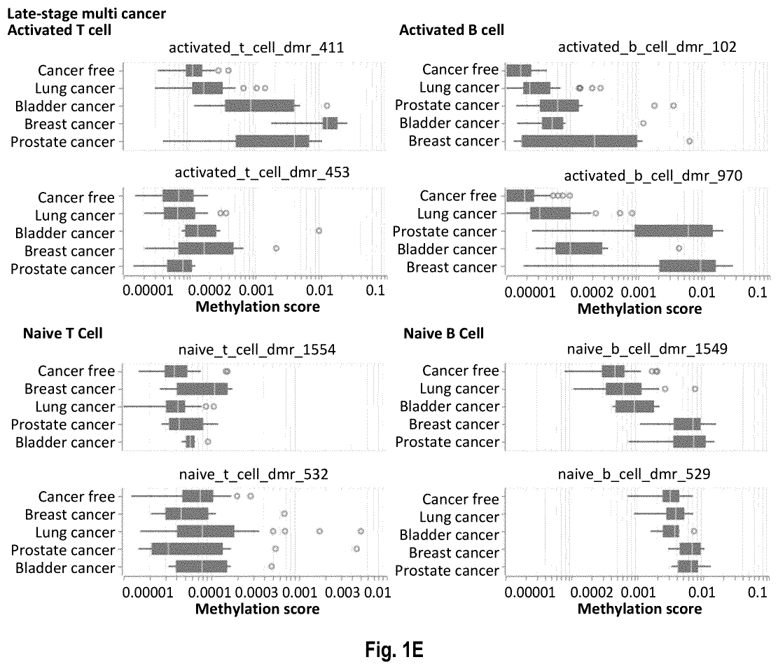

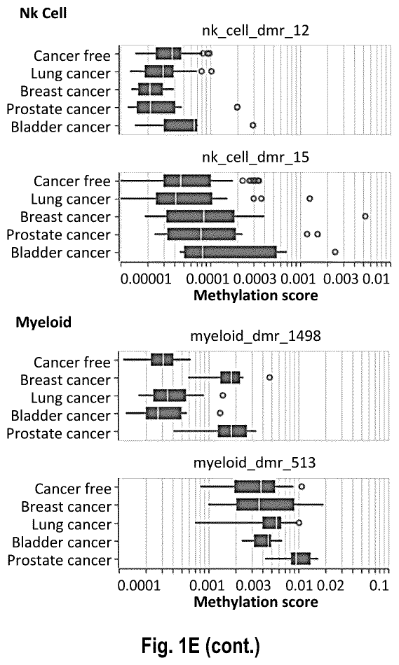

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. See, e.g., Liu et al., Nature Biotechnology 2019; 37:424-429 (e.g., at Supplementary FIG. 1 and Supplementary Note 7). Performing TAP conversion can facilitate identifying positions containing unmodified C using the sequence reads. This procedure encompasses Tet-assisted pyridine borane sequencing (TAPS), described in further detail in Liu et al. 2019, supra.

Alternatively, protection of hmC (e.g., using βGT) can be combined with Tet-assisted conversion with a substituted borane reducing agent. Performing such TAPSβ conversion can facilitate distinguishing positions containing unmodified C or hmC on the one hand from positions containing mC using the sequence reads. For an exemplary description of this type of conversion, see, e.g., Liu et al., Nature Biotechnology 2019; 37:424-429.

In some embodiments, the procedure that affects a first nucleobase in the DNA differently from a second nucleobase in the DNA comprises APOBEC-coupled epigenetic (ACE) conversion. Performing ACE conversion can facilitate distinguishing positions containing hmC from positions containing mC or unmodified C using the sequence reads. For an exemplary description of ACE conversion, see, e.g., Schutsky et al., Nature Biotechnology 2018; 36:1083-1090.

In some embodiments, procedure that affects a first nucleobase in the DNA differently from a second nucleobase in the DNA comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N⁶-methyladenine (mA), N⁶-hydroxymethyladenine (hmA), or N⁶-formyladenine (fA).

g. Captured Set; Target Regions

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell types but not to the extent observed in the one cell type. In some embodiments, the hypermethylation variable target regions show lower methylation in healthy cfDNA than in at least one other tissue type.

In some embodiments, a captured epigenetic target region set captured from a sample or second subsample comprises hypomethylation variable target regions. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell types but not to the extent observed in the one cell type. In some embodiments, the hypomethylation variable target regions show higher methylation in healthy cfDNA than in at least one other tissue type.

Without wishing to be bound by any particular theory, in an individual with cancer, proliferating or activated immune cells (and potentially also cancer cells) may shed more DNA into the bloodstream than immune cells in a healthy individual (and healthy cells of the same tissue type, respectively). As such, the distribution of cell type and/or tissue of origin of cfDNA may change upon carcinogenesis. For example, the distribution of immune cell type of origin may change in a subject having cancer, precancer, infection, transplant rejection, or other disease or disorder directly or indirectly affecting the immune system. The status of epigenetic target regions of certain immune cell types likewise may change in a subject having such a disease relative to a healthy subject or relative to the same subject prior to having the disease or disorder. Thus, variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Stunnenberg, H. G. et al., “The International Human Epigenome Consortium: A Blueprint for Scientific Collaboration and Discovery,” Cell 167, 1145 (2016) (doi.org/10.1186/s13059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set.

Where DNA (e.g., a sample or subsample) has been subjected to a procedure such as bisulfite conversion, treatment with a deaminase, or any of the other such procedures mentioned herein that alter the base-pairing specificity of certain bases, enrichment or capture may use oligonucleotides (e.g., primers or probes) specific for the altered or unaltered sequence, as desired.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2-to 1.4-fold greater concentration, a 1.4-to 1.6-fold greater concentration, a 1.6-to 1.8-fold greater concentration, a 1.8-to 2.0-fold greater concentration, a 2.0-to 2.2-fold greater concentration, a 2.2-to 2.4-fold greater concentration a 2.4-to 2.6-fold greater concentration, a 2.6-to 2.8-fold greater concentration, a 2.8-to 3.0-fold greater concentration, a 3.0-to 3.5-fold greater concentration, a 3.5-to 4.0, a 4.0-to 4.5-fold greater concentration, a 4.5-to 5.0-fold greater concentration, a 5.0-to 5.5-fold greater concentration, a 5.5-to 6.0-fold greater concentration, a 6.0-to 6.5-fold greater concentration, a 6.5-to 7.0-fold greater, a 7.0-to 7.5-fold greater concentration, a 7.5-to 8.0-fold greater concentration, an 8.0-to 8.5-fold greater concentration, an 8.5-to 9.0-fold greater concentration, a 9.0-to 9.5-fold greater concentration, 9.5-to 10.0-fold greater concentration, a 10-to 11-fold greater concentration, an 11-to 12-fold greater concentration a 12-to 13-fold greater concentration, a 13-to 14-fold greater concentration, a 14-to 15-fold greater concentration, a 15-to 16-fold greater concentration, a 16-to 17-fold greater concentration, a 17-to 18-fold greater concentration, an 18-to 19-fold greater concentration, a 19-to 20-fold greater concentration, a 20-to 30-fold greater concentration, a 30-to 40-fold greater concentration, a 40-to 50-fold greater concentration, a 50-to 60-fold greater concentration, a 60-to 70-fold greater concentration, a 70-to 80-fold greater concentration, a 80-to 90-fold greater concentration, or a 90-to 100-fold greater concentration. The degree of difference in concentrations accounts for normalization for the footprint sizes of the target regions, as discussed in the definition section.

### (a) Epigenetic Target Region Set

In some embodiments, an epigenetic target region set may comprise one or more types of target regions likely to differentiate DNA from different immune cell types and other non-immune cell types and/or to differentiate neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein.

In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### 1. Hypermethylation Variable Target Regions

In some embodiments, the epigenetic target region set comprises one or more hypermethylation variable target regions. In some embodiments, hypermethylation variable target regions are exclusively hypermethylated in one immune cell type or hypermethylated to a greater extent in one immune cell type than in any other immune cell type or than in any other immune cell type within the same immune cell cluster. In some such embodiments, hypermethylation variable target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypermethylation variable target regions that are useful for deconvoluting immune cell types may further change in certain disease states, such as cancer. Thus, in some embodiments, hypermethylation variable target regions that are useful for deconvoluting immune cell types are also useful for determining the likelihood that the subject from which the sample was obtained has cancer or precancer. In some such embodiments, hypermethylation variable target regions are useful for determining whether levels of particular immune cell types are abnormal and whether such abnormal levels are likely related to the presence of cancer or precancer, or if they are related to a different disease or condition other than cancer or precancer.

In some embodiments, certain hypermethylation variable target regions exhibit an increase in the level of observed methylation, e.g., are hypermethylated, in DNA produced by neoplastic cells, such as tumor or cancer cells. Detection of such hypermethylation variable target regions, e.g., in conjunction with detection of hypermethylation variable target regions indicative of immune cell types, may further increase the specificity and/or sensitivity of methods described herein. In some embodiments, such increases in observed methylation in hypermethylated variable target regions indicate an increased likelihood that a sample (e.g., of cfDNA) was obtained from a subject having cancer. For example, hypermethylation of promoters of tumor suppressor genes has been observed repeatedly. See, e.g., Kang et al., Genome Biol. 18:53 (2017) and references cited therein. In another example, as discussed above, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions useful for determining the likelihood that a subject has cancer are different than the hypermethylation variable target regions useful for determining the levels of particular immune cell types. In some embodiments, at least some of the hypermethylation variable target regions useful for determining the likelihood that a subject has cancer are the same as the hypermethylation variable target regions useful for determining the levels of particular immune cell types.

An extensive discussion of methylation variable target regions in colorectal cancer is provided in Lam et al., Biochim Biophys Acta. 1866:106-20 (2016). These include VIM, SEPT9, ITGA4, OSM4, GATA4 and NDRG4. An exemplary set of hypermethylation variable target regions based on colorectal cancer (CRC) studies is provided in Table 1. Many of these genes likely have relevance to cancers beyond colorectal cancer; for example, TP53 is widely recognized as a critically important tumor suppressor and hypermethylation-based inactivation of this gene may be a common oncogenic mechanism.

TABLE 1
Exemplary Hypermethylation Target
Regions based on CRC studies.

| Gene Name | Additional Gene Name | Chromosome |
|---|---|---|
| VIM | | chr10 |
| SEPT9 | | chr17 |
| CYCD2 | CCND2 | chr12 |
| TFPI2 | | chr7 |
| GATA4 | | chr8 |
| RARB2 | RARB | chr3 |
| p16INK4a | CDKN2A | chr9 |
| MGMT | | chr10 |
| APC | | chr5 |
| NDRG4 | | chr16 |
| HLTF | | chr3 |
| HPP1 | TMEFF2 | chr2 |
| hMLH1 | MLH1 | chr3 |
| RASSF1A | RASSF1 | chr3 |
| CDH13 | | chr16 |
| IGFBP3 | | chr7 |
| ITGA4 | | chr2 |

In some embodiments, the hypermethylation variable target regions comprise a plurality of loci listed in Table 1, e.g., at least 10%, 20%, 30%, 40%, 50%, 60%, 70%, 80%, 90%, or 100% of the loci listed in Table 1. For example, for each locus included as a target region, there may be one or more probes with a hybridization site that binds between the transcription start site and the stop codon (the last stop codon for genes that are alternatively spliced) of the gene, or in the promoter region of the gene. In some embodiments, the one or more probes bind within 300 bp of the transcription start site of a gene in Table 1, e.g., within 200 or 100 bp.

Methylation variable target regions in various types of lung cancer are discussed in detail, e.g., in Ooki et al., Clin. Cancer Res. 23:7141-52 (2017); Belinksy, Annu. Rev. Physiol. 77:453-74 (2015); Hulbert et al., Clin. Cancer Res. 23:1998-2005 (2017); Shi et al., BMC Genomics 18:901 (2017); Schneider et al., BMC Cancer. 11:102 (2011); Lissa et al., Transl Lung Cancer Res 5 (5): 492-504 (2016); Skvortsova et al., Br. J. Cancer. 94 (10): 1492-1495 (2006); Kim et al., Cancer Res. 61:3419-3424 (2001); Furonaka et al., Pathology International 55:303-309 (2005); Gomes et al., Rev. Port. Pneumol. 20:20-30 (2014); Kim et al., Oncogene. 20:1765-70 (2001); Hopkins-Donaldson et al., Cell Death Differ. 10:356-64 (2003); Kikuchi et al., Clin. Cancer Res. 11:2954-61 (2005); Heller et al., Oncogene 25:959-968 (2006); Licchesi et al., Carcinogenesis. 29:895-904 (2008); Guo et al., Clin. Cancer Res. 10:7917-24 (2004); Palmisano et al., Cancer Res. 63:4620-4625 (2003); and Toyooka et al., Cancer Res. 61:4556-4560, (2001).

An exemplary set of hypermethylation variable target regions based on lung cancer studies is provided in Table 2. Many of these genes likely have relevance to cancers beyond lung cancer; for example, Casp8 (Caspase 8) is a key enzyme in programmed cell death and hypermethylation-based inactivation of this gene may be a common oncogenic mechanism not limited to lung cancer. Additionally, a number of genes appear in both Tables 1 and 2, indicating generality.

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

Any of the foregoing embodiments concerning target regions identified in Table 2 may be combined with any of the embodiments described above concerning target regions identified in Table 1. In some embodiments, the hypermethylation variable target regions comprise a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, the hypermethylation variable target regions comprise regions of one or more genes listed in Table 2b, e.g. at least 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1050, 1000, 1100, 1150 or 1200 genes listed in Table 2b. Hypermethylation of these genes can be useful for detecting contributions from immune cells to a DNA sample. In some embodiments, the hypermethylation variable target regions comprise regions of a plurality of genes listed in Table 2b, e.g., at least 10%, 20%, 30%, 40%, 50%, 60%, 70%, 80%, 90%, or 100% of the genes listed in Table 2b. In some embodiments, the hypermethylation variable target regions comprise regions of all of the genes listed in Table 2b.

TABLE 2b
Exemplary genes comprising exemplary hypermethylation variable target regions
GENES

| | | | | | | |
|---|---|---|---|---|---|---|
| CCL5 | RSAD2 | DBNDD1 | CD80 | CD2 | ITPK1 | PRR5 |
| CD19 | ISG15 | SNRPB | CEACAM4 | G0S2 | JAG1 | PTGS2 |
| CD37 | MMP3 | MNAT1 | SH2D1A | MT1G | MMRN2 | PTPN11 |
| CD3D | MARCKSL1 | NUP35 | SAA2-SAA4 | CARD16 | NFIL3 | PVRIG |
| CD3E | IGF2R | TCEB1 | TMEM150B | MT1H | PDGFB | RELA |
| CD3G | LAMB1 | HSPB11 | PYHIN1 | SERPINA1 | PGPEP1 | RICTOR |
| CD3Z | SPARC | C19orf48 | CCL7 | ACP5 | RPL7A | RIPK2 |
| CD79A | FN1 | ID3 | PDCD1 | CXCL5 | SERPINB5 | SPP1 |
| CD79B | ITGA4 | IPO4 | BNC1 | CD74 | SERPINH1 | STAT4 |
| CD8A | SMO | FARSB | ICOS | C1QB | STC1 | TNFRSF14 |
| CD8B1 | MMP19 | EIF4G1 | ITK | HLA-DRB5 | TNFAIP6 | TNFRSF25 |
| IGHG3 | ITGB8 | SKA1 | CLEC4E | FADS3 | VCAN | TNFSF9 |
| IGJ | ITGA5 | MFSD11 | GZMK | IL15RA | VEGFA | TRAT1 |
| IGLC1 | NID1 | PLAUR | UBASH3A | MFI2 | VEGFB | TSLP |
| CD14 | TIMP1 | MARVELD2 | CRTAM | IL4I1 | VEGFC | VTCN1 |
| LCK | SEMA3F | MCM3 | EOMES | HLA-A | ATF3 | CCL13 |
| LTB | RHOQ | DHFR | P2RY10 | TM4SF19 | BATF3 | CCL14 |
| MS4A1 | CTNNB1 | RNF41 | NCF1 | MMP9 | CD1C | CCL18 |
| CORO1A | MMP2 | ID2 | CARD17 | ACY3 | CD36 | CCL19 |
| MNDA | SERPINE1 | H2AFZ | ZBP1 | KRT14 | CD4 | CCL20 |
| CCRL2 | EPHB2 | CDK2 | TNFRSF9 | MT1X | CDC20 | CCL21 |
| SLC7A7 | COL16A1 | NCLN | PDCD1LG2 | MT1M | CTSW | CCL22 |
| HLA-DMA | EPHA2 | ZWILCH | IL12RB1 | IFITM1 | CXCL1 | CCL3/L1 |
| FYB | TNC | DYNLT1 | LILRA6 | IFI35 | DTX3L | CSF1 |
| RNASE6 | JUP | C16orf61 | CXCR3 | BST2 | HLA-DMB | CSF2 |
| TLR2 | ITGA3 | SLC25A40 | SLAMF1 | CD8B | HLA-DOA | CSF2RB |
| CTSC | TCF7L2 | RHOC | SLAMF6 | BATF2 | HLA-DOB | CSF3R |
| LILRB4 | COL3A1 | CCT5 | RHOH | CTSZ | HLA-DPA1 | CX3CL1 |
| PSCDBP | CDH6 | PDIA4 | GPR114 | HAPLN3 | HLA-DPB1 | CX3CR1 |
| CTSS | WNT2B | SNRPA | SLA2 | TAP2 | HLA-DQA1 | CXCL12 |
| RASSF4 | ADAM9 | RBM14 | IL18RAP | ODF3B | HLA-DQA2 | CXCL14 |
| MSN | DSP | PDLIM7 | GPR171 | SQRDL | IRF8 | CXCL16 |
| CYBB | HSPG2 | PITPNC1 | SCIMP | DOK2 | KIF2C | CXCL2 |
| LAPTM5 | ARHGAP1 | TPM3 | P2RY13 | UBE2L6 | KIR2DL3 | CXCL3 |
| DOCK2 | ITGB5 | CORO1C | LAX1 | SLC15A3 | KIR3DL1 | CXCL6 |
| FCGR1A | IGFBP5 | ERLIN1 | AOAH | SECTM1 | KIR3DL2 | CXCL8 |
| EVI2B | ARHGDIA | PAICS | CXCL11 | GAL | KLRD1 | CXCR2 |
| ADCY7 | LRP1 | TPRKB | CD38 | CXCL13 | MRC1 | GNG4 |
| CD48 | IGFBP2 | SKA2 | GRAP2 | RAC2 | PSMB10 | IL10 |
| ARHGAP15 | CTNNA1 | MYBL1 | WDFY4 | IL1R2 | PSMB5 | IL11 |
| ARRB2 | LRRC17 | SH3BP5L | LTA | LRFN4 | PSMB8 | IL11RA |
| SYK | MMP14 | BRCA2 | GZMH | CLEC4A | PSMC4 | IL12RB2 |
| BTK | NEO1 | SAR1A | PTPN22 | SLAMF8 | THBD | IL16 |
| TNFAIP3 | EFNA5 | POLR3K | IRF4 | SH2D2A | TNF | IL1A |
| FCGR2A | ITGB3 | MRPS28 | GNLY | CDC25B | TRIM21 | IL1B |
| VSIG4 | EPHB3 | NUP107 | CCR5 | PSME2 | UBA7 | IL1RN |
| FPRL2 | CD44 | TUBG1 | SNX20 | GNGT2 | UBB | IL22RA1 |
| IL10RA | IGFBP4 | PNN | LILRB5 | TYROBP | UBE2C | IL24 |
| IFI16 | TNFRSF1A | FAM167A | RGS18 | AIF1 | ULBP2 | IL33 |
| ITGB2 | RAC1 | RFC3 | EMR1 | WARS | VHL | IL34 |
| IL7R | PXN | MYL6 | TIGIT | CD300A | AKT1 | IL6 |
| TBXAS1 | PLAT | MCM7 | SIGLEC7 | C1QC | APC | IL6R |
| FMNL1 | COL8A1 | MAGOHB | GPR65 | ARPC1B | BAD | JAK2 |
| FLI1 | WNT8B | FAM89B | TNFRSF17 | CIITA | BAX | JAK3 |
| RASSF2 | IGFBP3 | TOMM40 | ADAMDEC1 | HAVCR2 | BBC3 | PF4 |
| LYZ | RHOA | CDCA4 | HK3 | SPI1 | BCL2 | PIK3R5 |
| CD163 | EPHB4 | MT3 | LILRB2 | MIR3123 | BCL2L1 | SHC2 |
| CD97 | MMP1 | MTHFD1 | CTLA4 | ACAA2 | BCL6B | STAT2 |
| CCL2 | PAK1 | PSMD12 | SIGLEC12 | KRTAP19-8 | BID | STAT3 |
| FCGR2B | MTA1 | MYBL2 | CXorf21 | LPCAT1 | BIRC3 | CBLC |
| MERTK | THBS2 | CKLF | SPIB | CARTPT | BIRC5 | CD47 |
| CD84 | CSPG2 | NRIP3 | CLEC4G | ARHGAP26 | BLM | CNTFR |
| CD53 | MMP17 | EZR | CD300C | RPA3 | CASP3 | GHR |
| CD86 | CD59 | C12orf24 | CD300E | MIR129-1 | CASP8 | GZMM |
| HMHA1 | DVL3 | GPLD1 | LILRB1 | FAM150A | CASP9 | IFITM2 |
| CTSL | RHOB | SRM | LAG3 | KCNB2 | CDH1 | IRF9 |
| EVI2A | COL6A3 | RAB3B | HMGA2 | TERF1 | FADD | JAK1 |
| TNFRSF1B | NOTCH2 | NLN | VNN1 | KCNK9 | HMGB1 | KLRB1 |
| CXCR4 | BSG | MT1F | IL2RA | PTK2 | LY96 | KLRK1 |
| LCP1 | MMP11 | TNFRSF12A | NCR3 | GPR20 | RIPK1 | LIF |
| SAMHD1 | COL1A2 | TPI1 | CCR8 | TSNARE1 | RIPK3 | PRLR |
| CPVL | ZYX | HAS2 | TFEC | MIR4475 | ROCK1 | SIRPA |
| HLA-DRB1 | RND3 | APOO | CCR2 | IGFBPL1 | TICAM1 | SPRY4 |
| C13orf18 | THBS1 | FBXO41 | STAP1 | LOC100129316 | TLR3 | DDB2 |
| GIMAP4 | RHOG | MRPL37 | IDO1 | QRFP | TLR4 | ERCC3 |
| SAMSN1 | ICAM1 | GSTCD | MYO1G | CAMSAP1 | TNFRSF10B | FANCA |
| PLCG2 | LAMA4 | SDC1 | SIT1 | GPR37L1 | TNFSF10 | MGMT |
| OSBPL3 | DVL1 | WDR54 | CD209 | CCDC85C | TP53 | NEIL1 |
| RUNX3 | PAK2 | RNF138 | LILRA5 | GACAT2 | BNIP3 | PARP4 |
| FCGR3A | COL6A1 | APITD1 | SIGLEC1 | LINC01443 | DEPTOR | POLR2A |
| AMPD3 | FGD1 | RMND5B | UBD | CDH2 | HIF1A | TNKS |
| MYO1F | CEP78 | ENO1 | SPN | MIR4436A | HRAS | UBE2T |
| CECR1 | LSM3 | MAP3K8 | CXCL9 | INSM1 | KRAS | XCL1 |
| LYN | LRRC40 | TMEM130 | CD70 | LOC101929625 | MAP3K7 | XCL2 |
| MPP1 | STK17A | SNX17 | GZMB | COL18A1 | MAPK10 | ARID1A |
| LRMP | RPN1 | KRR1 | SIGLEC10 | OPA1-AS1 | MTOR | BRD3 |
| FGL2 | JUNB | TAGLN | CD300LF | C9 | NRAS | BRD4 |
| NCKAP1L | NUP85 | PA2G4 | CCL8 | MIR585 | PIK3CA | DNMT1 |
| HCLS1 | FLNC | RUVBL1 | PTPN7 | MAD1L1 | PIK3CD | HDAC11 |
| SELL | HMGN2 | SNRPD1 | CCL4 | CPA2 | PIK3R1 | HDAC3 |
| CASP1 | RPP40 | LOXL2 | CMKLR1 | LOC100130298 | PIK3R2 | HDAC4 |
| SELPLG | UQCR10 | POLE2 | ICAM3 | C8orf34 | PRKAA2 | HDAC5 |
| CD33 | AIMP2 | MAPRE1 | SIRPG | OC90 | PRKACB | HELLS |
| GPNMB | CHEK1 | IMP4 | NLRP3 | TRAPPC9 | PTEN | HMGA1 |
| NCF2 | VTA1 | EMP2 | MARCO | MED27 | RPS6KB1 | KAT2B |
| FNBP1 | EXOSC8 | PSMD2 | CCL4L1 | MLH1 | RPTOR | MAP3K12 |
| IL18 | CENPO | MET | CD180 | MSH2 | ANLN | BMP2 |
| B2M | PNO1 | IFRD2 | SIGLEC9 | MSH6 | ATM | GAS1 |
| SP140 | SLC16A1 | LMNB2 | KRT6B | PMS2 | CCNA1 | GLI1 |
| FCER1G | WDR77 | PLOD2 | NFAM1 | BRCA1 | CCNB1 | IHH |
| LCP2 | UBE2J1 | NCEH1 | GPR84 | TERT | CCND1 | PRKX |
| LY86 | NOP16 | NME1 | KLK5 | EphA2 | CCND3 | WNT10A |
| LAIR1 | NUDT1 | STRA13 | CCL23 | cGAS | CCNO | WNT11 |
| IFI30 | SMC2 | ACTL6A | ZBED2 | STING | CDC25C | WNT2 |
| TNFSF13B | SLC25A5 | DLEU1 | CD28 | CD8 | CDK6 | WNT3A |
| LST1 | NUPL1 | SNRPA1 | CEACAM21 | PDL2 | CDKN1A | WNT4 |
| FGR | DLEU2 | CBX1 | PRF1 | ASXL3 | CDKN1C | WNT5A |
| NPL | PDAP1 | LYAR | CSF3 | SPEDF | CDKN2A | WNT5B |
| PLEK | CCBL2 | PTPLB | KIAA0226L | PSMA | CDKN2B | WNT7B |
| PTPRC | COX17 | PFN1 | CXCR6 | GPC3 | CENPF | ALDOA |
| GNPTAB | BCCIP | CENPJ | CCL24 | RAD51 | CEP55 | EGF |
| SLC1A3 | PLG | COTL1 | ARHGAP9 | ACVR1 | EXO1 | EGFR |
| HCK | RGS8 | SPRYD7 | LILRB3 | APLNR | H2AFX | ERBB2 |
| NPC2 | SNRPC | USPL1 | VNN2 | CDC27 | MELK | HK1 |
| C3AR1 | PLK4 | MRPL12 | ZAP70 | CDK4 | MKI67 | HK2 |
| PIK3CG | NUTF2 | ADAMTS1 | TNFSF8 | HLA-C | MYC | IFNGR1 |
| DAPK1 | LSM4 | GLRX3 | HEPHL1 | HLA-E | NBN | IFNGR2 |
| ALOX5AP | SMS | WSB2 | CCL3 | HLA-G | POLD1 | LDHA |
| CSF1R | EBNA1BP2 | MRPS16 | CLEC10A | IL1R | PRKCA | NOS2 |
| CUGBP2 | C13orf27 | DCLRE1B | CCL3L3 | IL2RB | RAD50 | PDK1 |
| APOE | VDAC1 | MKKS | CCR4 | INFGR1 | RAD51C | PFKFB3 |
| APOC1 | PSMD14 | C3orf26 | ITGAL | INFGR2 | RB1 | SLC2A1 |
| CD52 | MYCBP | CPEB4 | SAA2 | IRF1 | RBL2 | TFRC |
| LHFPL2 | SMURF2 | SPAG17 | GZMA | IRF2 | RRM2 | CD276 |
| C1orf54 | GNG11 | MLF1IP | BCL2A1 | KIR2DS2 | TYMS | CD6 |
| IKZF1 | F3 | UAP1 | RNASE2 | KIR2DS3 | ADORA2A | CDH5 |
| SH2B3 | BRIP1 | COQ2 | SAA1 | PIAS4 | BTLA | CLEC14A |
| WIPF1 | HNRNPA2B1 | WDHD1 | NKG7 | PTPN2 | CD247 | CLEC5A |
| IFI44 | DCK | DCBLD2 | MT2A | SERPINB3 | CD27 | CLEC7A |
| IFI44L | ALKBH7 | KIAA0090 | FAM26F | SERPINB4 | CD274 | CLECL1 |
| DDX58 | HN1L | SAR1B | PSMB9 | SPOP | CD40 | ICAM2 |
| IFI6 | TPM1 | PSMA7 | TYMP | TAPBP | CD40LG | ICAM5 |
| IFI27 | HYLS1 | PSMC3 | CD7 | TGFBR1 | CD69 | ITGA1 |
| IFIT2 | HAUS1 | COPS6 | SYT12 | CD58 | CHUK | ITGA2 |
| IFIT1 | NUP93 | DUT | S100A7 | ANGPT1 | DPP4 | ITGAE |
| IFIT3 | SNRPE | PPIH | HLA-F | ANGPT2 | EGR1 | ITGAM |
| CXCL10 | ITGA6 | PHF19 | MT1E | ANGPTL4 | FYN | ITGAX |
| MX1 | CENPN | TPM2 | ISG20 | CCND2 | ICOSLG | NCAM1 |
| OAS1 | C11orf24 | MCTS1 | C1QA | CCNE1 | IKBKB | NECTIN1 |
| OAS2 | GGH | EIF4EBP1 | TNFRSF18 | CES3 | IKBKG | PECAM1 |
| OAS3 | PFKP | HNRNPR | IL32 | DLL4 | IL15 | PVR |
| HERC5 | FARSA | IFNG | SOCS1 | E2F3 | IL17A | SELE |
| SAMD9 | EIF2AK1 | TLR8 | HLA-DQB1 | EDN1 | IL18R1 | SELP |
| HERC6 | CENPW | FASLG | IFITM3 | EZH2 | IL2 | THY1 |
| DDX60 | TUBA4A | KIR2DL4 | PI3 | FGF18 | IL4 | VCAM1 |
| RTP4 | TRA2B | IL21R | HCST | FGFR1 | LY9 | EIF2AK2 |
| IFIH1 | UMPS | GBP5 | SUSD3 | FLT1 | NECTIN2 | FLNB |
| STAT1 | MRTO4 | ZNF683 | HLA-B | FSTL3 | NFATC2 | GBP1 |
| TAP1 | NUDT15 | PLA2G2D | IL2RG | HEY1 | NFKB1 | GBP2 |
| OASL | PGM2 | TBX21 | HLA-DRA | ITGAV | NFKBIA | GBP4 |
| TPSD1 | GATA2 | SERPINF1 | FCER1A | DAPP1 | CCR7 | EREG |
| SLC18A2 | HPGDS | PLAC8 | CD1A | C10ORF54 | XCR1 | CD81 |
| IL1RL1 | TCF4 | JCHAIN | AXL | ANPEP | MTSS1 | PRDM1 |
| VWA5A | MZB1 | SLC15A4 | CD1E | CST7 | MSA4A7 | ABL2 |
| TPSAB2 | IL3RA | ITM2C | PKIB | FLT3 | LILRA1 | SDC4 |
| IFNA1 | PDGFA | MMP7 | CRABP2 | TNFRSF4 | FZD9 | PNOC |
| IFNAR1 | PDGFRB | NID2 | DAB2 | TNFSF12 | GPC4 | HLA-DR |
| IRF3 | PLA1A | RELN | FCAR | TNFSF13 | SFRP1 | LIN |
| IRF5 | PLA2G2A | AQP9 | FCN1 | TNFSF18 | SFRP4 | CD11b |
| IRF7 | RASAL1 | CEBPB | FOSL1 | TNFSF4 | SOX11 | CD15 |
| BLK | RASGRF1 | ERO1A | FPR1 | APH1B | SOX2 | CD11c |
| CD5 | RELB | FBP1 | FPR3 | DLL1 | FOXP3 | CD34 |
| CD96 | TGFB1 | G6PD | IER3 | DTX4 | NCR1 | CD83 |
| F2RL1 | TGFB2 | GLS | OLR1 | HES1 | CEACAM3 | PD-L1 |
| SLAMF7 | TGFB3 | GOT1 | PDZK1IP1 | JAG2 | FCGR3A/B | CD45 |
| DUSP1 | TGFBR2 | GOT2 | S100A12 | MAML2 | SIGLEC5 | CD124 |
| DUSP2 | A2M | LDHB | S100A8 | MFNG | CPA3 | CD66 |
| DUSP5 | COL11A1 | PC | S100A9 | NOTCH1 | HDC | CD3 |
| FAS | COL11A2 | PCK2 | SIGLEC8 | TCL1A | MS4A2 | CD13 |
| FGF13 | COL17A1 | PFKM | SIRPB2 | ACVR1C | TPSAB1/B2 | TPSAB1 |
| FGF9 | COL4A5 | PKM | SLC11A1 | BAMBI | CD68 | LILRA4 |
| KDR | COL5A1 | SGK1 | TLR1 | ID4 | MS4A4A | CD16 |
| KIT | COMP | SLC1A5 | TREM1 | INHBA | CD244 | PLTP |
| MAP3K5 | LAMA1 | SLC7A5 | TREM2 | SMAD5 | PTGER4 | ADM |
| NF1 | LAMB3 | AREG | NFKBIE | AXIN1 | HSD11B1 | CSTB |
| NFKB2 | LAMC2 | ARG1 | TNFRSF11A | DKK1 | FAM30A | PPARG |
| NGFR | LTBP1 | C5AR1 | TNFRSF11B | FZD8 | FCRL2 | RGCC |

Additional hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, where different epigenetic target regions are captured from first and second subsamples, the epigenetic target regions captured from the first subsample comprise hypermethylation variable target regions.

a. Hypomethylation Variable Target Regions

In some embodiments, the epigenetic target region set comprises one or more hypomethylation variable target regions. In some embodiments, hypomethylation variable target regions are exclusively hypomethylated in one immune cell type or hypomethylated to a greater extent in one immune cell type than in any other immune cell type or in any other immune cell type within the same immune cell cluster. In some such embodiments, hypomethylation variable target regions indicate the levels of particular immune cell types from which the DNA originated, including rare immune cell types such as activated B cells (including memory B cells and plasma cells), activated T cells (including regulatory T cells (Tregs), CD4 effector memory T cells, CD4 central memory T cells, CD8 effector memory T cells, and CD8 central memory T cells), and natural killer (NK) cells. Methylation patterns of hypomethylation variable target regions that are useful for deconvoluting immune cell types may further change in certain disease states, such as cancer. Thus, in some embodiments, hypomethylation variable target regions that are useful for deconvoluting immune cell types are also useful for determining the likelihood that the subject from which the sample was obtained has cancer or precancer. In some such embodiments, hypomethylation variable target regions are useful for determining whether levels of particular immune cell types are abnormal and whether such abnormal levels are likely related to the presence of cancer or precancer, or if they are related to a different disease or condition other than cancer or precancer.

Additionally, global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINEI elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions in which a decrease in the level of observed methylation indicates an increased likelihood of the presence of cancer. Detection of such hypomethylation variable target regions, e.g., in conjunction with detection of hypomethylation variable target regions indicative of immune cell types, may further increase the specificity and/or sensitivity of methods described herein. In another example, as discussed above, hypomethylation variable target regions can include regions that do not necessarily differ in methylation in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions useful for determining the likelihood that a subject has cancer are different than the hypomethylation variable target regions useful for determining the levels of particular immune cell types. In some embodiments, at least some of the hypomethylation variable target regions useful for determining the likelihood that a subject has cancer are the same as the hypomethylation variable target regions useful for determining the levels of particular immune cell types.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

Additionally, hypomethylation target regions may be obtained, e.g., from Fox-Fisher et al., Elife November 29; 10 (2021), EpiDISH R package, Moss et al., Nat Commun 9:1 (2018), and Loyfer et al. bioRxiv https://doi.org/10.1101/2022.01.24.477547 (2022). In some embodiments, the hypomethylation target regions can be specific to one or more types of immune cells.

In some embodiments, where different epigenetic target regions are captured from first and second subsamples, the epigenetic target regions captured from the second subsample comprise hypomethylation variable target regions. In some embodiments, the epigenetic target regions captured from the second subsample comprise hypomethylation variable target regions and the epigenetic target regions captured from the first subsample comprise hypermethylation variable target regions.

b. CTCF Binding Regions

CTCF is a DNA-binding protein that contributes to chromatin organization and often colocalizes with cohesin. Perturbation of CTCF binding sites has been reported in a variety of different cancers. See, e.g., Katainen et al., Nature Genetics, doi: 10.1038/ng.3335, published online 8 Jun. 2015; Guo et al., Nat. Commun. 9:1520 (2018). CTCF binding results in recognizable patterns in cfDNA that can be detected by sequencing, e.g., through fragment length analysis. Details regarding sequencing-based fragment length analysis are provided in Snyder et al., Cell 164:57-68 (2016); WO 2018/009723; and US20170211143A1, each of which are incorporated herein by reference.

Thus, perturbations of CTCF binding result in variation in the fragmentation patterns of cfDNA. As such, CTCF binding sites are a type of fragmentation variable target regions.

There are many known CTCF binding sites. See, e.g., the CTCFBSDB (CTCF Binding Site Database), available on the Internet at insulatordb.uthsc.edu/; Cuddapah et al., Genome Res. 19:24-32 (2009); Martin et al., Nat. Struct. Mol. Biol. 18:708-14 (2011); Rhee et al., Cell. 147:1408-19 (2011), each of which are incorporated by reference. Exemplary CTCF binding sites are at nucleotides 56014955-56016161 on chromosome 8 and nucleotides 95359169-95360473 on chromosome 13.

Accordingly, in some embodiments, the epigenetic target region set includes CTCF binding regions. In some embodiments, the CTCF binding regions comprise at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above.

In some embodiments, at least some of the CTCF sites can be methylated or unmethylated, wherein the methylation state is correlated with the whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the CTCF binding sites.

c. Transcription Start Sites

Transcription start sites may also show perturbations in neoplastic cells. For example, nucleosome organization at various transcription start sites in healthy cells of the hematopoietic lineage—which contributes substantially to cfDNA in healthy individuals—may differ from nucleosome organization at those transcription start sites in neoplastic cells. This results in different cfDNA patterns that can be detected by sequencing, as discussed generally in Snyder et al., Cell 164:57-68 (2016); WO 2018/009723; and US20170211143A1. In another example, transcription start sites may not necessarily differ epigenetically in cancerous tissue relative to DNA from healthy tissue of the same type, but do differ epigenetically (e.g., with respect to nucleosome organization) relative to cfDNA that is typical in healthy subjects. Where, for example, the presence of a cancer results in increased cell death, such as apoptosis, of cells of the tissue type corresponding to the cancer, such a cancer can be detected at least in part using such differences in transcription start sites.

Thus, perturbations of transcription start sites also result in variation in the fragmentation patterns of cfDNA. As such, transcription start sites are also a type of fragmentation variable target regions.

Human transcriptional start sites are available from DBTSS (DataBase of Human Transcription Start Sites), available on the Internet at dbtss.hgc.jp and described in Yamashita et al., Nucleic Acids Res. 34 (Database issue): D86-D89 (2006), which is incorporated herein by reference.

Accordingly, in some embodiments, the epigenetic target region set includes transcriptional start sites. In some embodiments, the transcriptional start sites comprise at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, at least some of the transcription start sites can be methylated or unmethylated, wherein the methylation state is correlated with whether or not the cell is a cancer cell. In some embodiments, the epigenetic target region set comprises at least 100 bp, at least 200 bp, at least 300 bp, at least 400 bp, at least 500 bp, at least 750 bp, at least 1000 bp upstream and downstream regions of the transcription start sites.

d. Focal Amplifications

Although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set and may comprise one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

e. Methylation Control Regions

It can be useful to include control regions to facilitate data validation. In some embodiments, the epigenetic target region set includes control regions that are expected to be methylated or unmethylated in essentially all samples, regardless of whether the DNA is derived from a cancer cell or a normal cell. In some embodiments, the epigenetic target region set includes control hypomethylated regions that are expected to be hypomethylated in essentially all samples. In some embodiments, the epigenetic target region set includes control hypermethylated regions that are expected to be hypermethylated in essentially all samples.

### 2. Sequence-Variable Target Region Set

In some embodiments, the sequence-variable target region set comprises a plurality of regions known to undergo somatic mutations (e.g., single nucleotide variations and/or indels) in cancer. The single nucleotide variations and/or indels may be relative to a reference sequence, e.g., a published human genome sequence, such as the GRCh38 human genome assembly.

In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, or 70 of the SNVs of Table 3. In some embodiments, a sequence-variable target region set used in the methods of the present disclosure comprises at least 1, at least 2, at least 3, at least 4, at least 5, or 6 of the fusions of Table 3. In some embodiments, a sequence-variable target region set used in the methods of the present disclosure comprise at least a portion of at least 1, at least 2, or 3 of the indels of Table 3. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, a sequence-variable target region set used in the methods of the present disclosure comprises at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, a sequence-variable target region set used in the methods of the present disclosure comprises at least 1, at least 2, at least 3, at least 4, at least 5, or 6 of the fusions of Table 4. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. Each of these genomic locations of interest may be identified as a backbone region or hot-spot region for a given panel. An example of a listing of hot-spot genomic locations of interest may be found in Table 5. In some embodiments, a sequence-variable target region set used in the methods of the present disclosure comprises at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5. Each hot-spot genomic region is listed with several characteristics, including the associated gene, chromosome on which it resides, the start and stop position of the genome representing the gene's locus, the length of the gene's locus in base pairs, the exons covered by the gene, and the critical feature (e.g., type of mutation) that a given genomic region of interest may seek to capture.

TABLE 3

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 (total | | |
| | | | | target | | |
| | | | | region) | | |
| | | | | 16330 (total | | |
| | | | | probe | | |
| | | | | coverage) | | |

Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1.

In some embodiments, the sequence-variable target region set comprises target regions from at least 10, 20, 30, or 35 cancer-related genes, such as the cancer-related genes listed above.

### B. Subjects

In some embodiments, the DNA (e.g., cfDNA) is obtained from a subject having a cancer or a precancer, an infection, transplant rejection, or other disease directly or indirectly affecting the immune system. In some embodiments, the DNA (e.g., cfDNA) is obtained from a subject suspected of having a cancer or a precancer, an infection, transplant rejection, or other disease directly or indirectly affecting the immune system. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

C. Pooling of DNA from Samples or Subsamples or Portions Thereof.

In some embodiments, the methods herein comprise preparing one or more pools comprising tagged DNA from a plurality of partitioned subsamples. In some embodiments, a pool comprises at least a portion of the DNA of a hypomethylated partition and at least a portion of the DNA of a hypermethylated partition. Target regions, e.g., including epigenetic target regions and/or sequence-variable target regions, may be captured from a pool. The steps of capturing a target region set from at least an aliquot or portion of a sample or subsample described elsewhere herein encompass capture steps performed on a pool comprising DNA from first and second subsamples. A step of amplifying DNA in a pool may be performed before capturing target regions from the pool. The capturing step may have any of the features described for capturing steps elsewhere herein.

In some embodiments, the methods comprise preparing a first pool comprising at least a portion of the DNA of a hypomethylated partition. In some embodiments, the methods comprise preparing a second pool comprising at least a portion of the DNA of a hypermethylated partition. In some embodiments, the methods comprise capturing at least a first set of target regions from the first pool, wherein the first set comprises sequence-variable target regions. A step of amplifying DNA in the first pool may be performed before this capture step. In some embodiments, capturing the first set of target regions from the first pool comprises contacting the DNA of the first pool with a first set of target-specific probes, wherein the first set of target-specific probes comprises target-binding probes specific for the sequence-variable target regions. In some embodiments, the methods comprise capturing a second plurality of sets of target regions from the second pool, wherein the second plurality comprises sequence-variable target regions and epigenetic target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions.

In some embodiments, sequence-variable target regions are captured from a second portion of a partitioned subsample. The second portion may include some, a majority, substantially all, or all of the DNA of the subsample that was not included in the pool. The regions captured from the pool and from the subsample may be combined and analyzed in parallel.

The epigenetic target regions may show differences in methylation levels and/or fragmentation patterns depending on whether they originated from a particular cell or tissue type or from a tumor or from healthy cells, as discussed elsewhere herein. The sequence-variable target regions may show differences in sequence depending on whether they originated from a tumor or from healthy cells.

Analysis of epigenetic target regions from a hypomethylated partition may be less informative in some applications than analysis of sequence-variable target regions from hypermethylated and hypomethylated partitions and epigenetic target regions from a hypermethylated partition. As such, in methods where sequence-variable target regions and epigenetic target regions are being captured, the latter may be captured to a lesser extent than one or more of the sequence-variable target regions are captured from the hypermethylated and hypomethylated partitions and/or to a lesser extent that epigenetic target regions are captured from a hypermethylated partition. For example, sequence-variable target regions can be captured from a portion of a hypomethylated partition that is not pooled with a hypermethylated partition, and the pool can be prepared with some (e.g., a majority, substantially all, or all) of the DNA from a hypermethylated partition and none or some (e.g., a minority) of the DNA from a hypomethylated partition. Such approaches can reduce or eliminate sequencing of epigenetic target regions from hypomethylated partitions, thereby reducing the amount of sequencing data that suffices for further analysis.

In some embodiments, including a minority of the DNA of a hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of a hypomethylated partition, e.g., less than about 50% of the DNA of a hypomethylated partition, such as less than or equal to about 45%, 40%, 35%, 30%, 25%, 20%, 15%, 10%, or 5% of the DNA of a hypomethylated partition. In some embodiments, the pool comprises about 5%-25% of the DNA of a hypomethylated partition. In some embodiments, the pool comprises about 10%-20% of the DNA of a hypomethylated partition. In some embodiments, the pool comprises about 10% of the DNA of a hypomethylated partition. In some embodiments, the pool comprises about 15% of the DNA of a hypomethylated partition. In some embodiments, the pool comprises about 20% of the DNA of a hypomethylated partition.

In some embodiments, the pool comprises a portion of a hypermethylated partition, which may be at least about 50% of the DNA of a hypermethylated partition. For example, the pool may comprise at least about 55%, 60%, 65%, 70%, 75%, 80%, 85%, 90%, or 95% of the DNA of a hypermethylated partition. In some embodiments, the pool comprises 50-55%, 55-60%, 60-65%, 65-70%, 70-75%, 75-80%, 80-85%, 85-90%, 90-95%, or 95-100% of the DNA of a hypermethylated partition. In some embodiments, the second pool comprises all or substantially all of the DNA of a hypermethylated partition.

In some embodiments, a first pool comprises substantially all or all of the DNA of a hypomethylated partition (e.g., wherein a second pool does not comprise DNA of a hypomethylated partition. In some embodiments, the second pool does not comprise DNA of a hypomethylated partition (e.g., wherein the first pool comprises substantially all or all of the DNA of a hypomethylated partition).

In some embodiments, a second pool comprises a portion of a hypermethylated partition, which may be any of the values and ranges set forth above with respect to a hypomethylated partition. In some embodiments, the second pool comprises all or substantially all of the DNA of a hypermethylated partition.

In an exemplary embodiment, after partitioning, the partitions separately undergo end repair and ligation to adapters comprising molecular barcodes and are then amplified separately. After the amplification, amplified molecules are enriched (still keeping the partitions separate). Post-enrichment, the enriched DNA are pooled according to any of the embodiments described herein, and then amplified again. After amplification, the molecules are sequenced.

In various embodiments, the methods further comprise sequencing the captured DNA, e.g., to different degrees of sequencing depth for the epigenetic and sequence-variable target region sets, consistent with the discussion above.

### D. Sequencing

In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms.

In some embodiments, sequencing comprises detecting and/or distinguishing unmodified and modified nucleobases. For example, PacBio sequencing (e.g., single-molecule real-time (SMRT) sequencing) offers the ability to directly detect of, e.g., 5-methylcytosine and 5-hydroxymethylcytosine as well as unmodified cytosine. See, e.g., Schatz., Nature Methods. 14 (4): 347-348 (2017); and U.S. Pat. No. 9,150,918. Also, Oxford nanopore sequencing systems (e.g., MinION sequencer) that can directly detect methylation of DNA (for example: 5-methylcytosine and 5-hydroxymethylcytosine) can be used here. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously. Similarly, Ion Torrent sequencing may also be used to directly detect methylation. Thus, in some embodiments, methylation status can be determined during sequencing, e.g., without or independently of a partitioning step or a conversion procedure such as bisulfite treatment.

The sequencing reactions can be performed on one or more forms of nucleic acids, such as those known to contain markers of cancer or of other disease. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can performed on at least 5, 10, 20, 70, 100, 200 or 500 different genes, or at most 5000, 2500, 1000, 500 or 100 different genes.

Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

### 1. Differential Depth of Sequencing

In some embodiments, nucleic acids corresponding to a sequence-variable target region set are sequenced to a greater depth of sequencing than nucleic acids corresponding to an epigenetic target region set. For example, the depth of sequencing for nucleic acids corresponding to sequence variant target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25-to 1.5-, 1.5-to 1.75-, 1.75-to 2-, 2-to 2.25-, 2.25-to 2.5-, 2.5-to 2.75-, 2.75-to 3-, 3-to 3.5-, 3.5-to 4-, 4-to 4.5-, 4.5-to 5-, 5-to 5.5-, 5.5-to 6-, 6-to 7-, 7-to 8-, 8-to 9-, 9-to 10-, 10-to 11-, 11-to 12-, 13-to 14-, 14-to 15-fold, or 15-to 100-fold greater, than the depth of sequencing for nucleic acids corresponding to an epigenetic target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater.

In some embodiments, DNA corresponding to a sequence-variable target region set, and/or to an epigenetic target region set are sequenced concurrently, e.g., in the same sequencing cell (such as the flow cell of an Illumina sequencer) and/or in the same composition, which may be a combined or pooled composition resulting from recombining separately captured sets or a composition obtained by, e.g., capturing the cfDNA corresponding to the sequence-variable target region set, and/or the captured cfDNA corresponding to an epigenetic target region set in the same vessel.

### E. Analysis

In some embodiments, a method described herein comprises determining the levels of particular immune cell types from which DNA originated. The immune cell types may comprise naïve and activated lymphocytes, myeloid cells at different points of differentiation, and/or other types described elsewhere herein. In some methods, the determination of levels of immune cell types facilitates determination of the likelihood that the subject from which the DNA was obtained has a disease or disorder related to the immune system, such as an infection, transplant rejection, or cancer or precancer.

In some embodiments, a method described herein comprises identifying the presence of DNA produced by a tumor (or neoplastic cells, or cancer cells) or by precancer cells. In some embodiments, a method described herein comprises identifying the presence of DNA produced by immune cells that are not tumor cells, cancer cells, or precancer cells. In some such embodiments, determination of immune cell distribution facilitates detection or diagnosis or cancer or precancer, or determination of cancer prognosis or cancer treatment options. For example, determining the ratios of different immune cell types may facilitate such detection or determination. In some embodiments, the ratio numerator is the number or relative number of neutrophils, monocytes, or both, and the ratio denominator is the number or relative number of T cells, B cells, NK cells, or all lymphocytes. In some embodiments, the ratio numerator is the number or relative number of neutrophils, and the ratio denominator is the number or relative number of T cells, B cells, NK cells, or all lymphocytes. In some embodiments, the ratio numerator is the number or relative number of monocytes, and the ratio denominator is the number or relative number of T cells, B cells, NK cells, or all lymphocytes. In some embodiments, the ratio numerator is the number or relative number of neutrophils and monocytes, and the ratio denominator is the number or relative number of T cells, B cells, NK cells, or all lymphocytes. In some embodiments, the ratio is a neutrophil to lymphocyte ratio. In some embodiments, the ratio is a monocyte to T cell ratio. In some embodiments, elevations in such ratios are associated with cancer. In other embodiments, reductions in such ratios are associated with cancer

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer or precancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, or other mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

An exemplary method for identification of immune cell types through NGS comprises the following steps:
1. Preparing an extracted DNA sample (e.g., extracted blood plasma DNA from a human sample) by ligating adapters comprising molecular tags to the DNA.
2. Partitioning adapter-ligated DNA into a plurality of differentially methylated subsamples by contacting the DNA with an agent that recognizes a modified cytosine, such as methyl cytosine, in the DNA.
4. Capturing hypermethylated variable target regions and hypomethylated variable target regions from the partitioned subsamples by contacting the subsamples with target-specific probes.
5. Enriching and/or eluting DNA comprising the target regions.
6. Amplifying the DNA and assaying in multiplex on an NGS instrument.
7. Analyzing NGS data, with the molecular tags being used to identify unique molecules.

An exemplary workflow is shown in FIG. 1A, wherein cfDNA obtained from a sample is partitioned based on the level of methylation of the cfDNA molecules. Molecular barcodes are then added to the cfDNA molecules of each partition, then epigenetic and optionally sequence-variable target regions are then captured from partitioned subsamples, providing a targeted library. The epigenetic target regions include hypermethylation variable target regions and/or hypomethylation variable target regions that are differentially methylated in certain types of immune cells. The targeted library may be amplified before sequencing, then sequencing provides sequence information used to determine levels of immune cell types and/or likelihood of the presence of a disease or disorder.

In some embodiments of methods described herein, molecular tags consist of nucleotides that are not altered by a procedure that affects a first nucleobase in the DNA differently from a second nucleobase in the DNA, such as any of those described herein (e.g., mC along with A, T, and G where the procedure is bisulfite conversion or any other conversion that does not affect mC; hmC along with A, T, and G where the procedure is a conversion that does not affect hmC;

etc.). In some embodiments of methods described herein, the molecular tags do not comprise nucleotides that are altered by a procedure that affects a first nucleobase in the DNA differently from a second nucleobase in the DNA, such as any of those described herein (e.g., the tags do not comprise unmodified C where the procedure is bisulfite conversion or any other conversion that affects C; the tags do not comprise mC where the procedure is a conversion that affects mC; the tags do not comprise hmC where the procedure is a conversion that affects hmC; etc.).

### II. Additional Features of Certain Disclosed Methods

### A. Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

A sample can comprise various amount of nucleic acid that contains genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., precancer cells or cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of cell-free nucleic acids in a sample before amplification range from about 1 fg to about 1 ug, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng-

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, IRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as C 1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA, and single stranded RNA. In some embodiments, single stranded DNA and RNA can be converted to double stranded forms so they are included in subsequent processing and analysis steps.

DNA molecules can be linked to adapters at either one end or both ends. Typically, double-stranded molecules are blunt ended by treatment with a polymerase with a 5′-3′ polymerase and a 3 ‘-5’ exonuclease (or proof-reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### B. Amplification

Sample nucleic acids flanked by adapters can be amplified by PCR and other amplification methods. Amplification is typically primed by primers that anneal or bind to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication. In some embodiments, the present methods perform dsDNA ligations with T-tailed and C-tailed adapters, which result in amplification of at least 50, 60, 70 or 80% of double stranded nucleic acids before linking to adapters. Preferably the present methods increase the amount or number of amplified molecules relative to control methods performed with T-tailed adapters alone by at least 10, 15 or 20%.

### C. Tags

Tags comprising barcodes can be incorporated into or otherwise joined to adapters. Tags can be incorporated by ligation, overlap extension PCR among other methods.

### 1. Molecular Tagging Strategies

Molecular tagging refers to a tagging practice that allows one to differentiate among DNA molecules from which sequence reads originated. Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by U.S. patent applications 20010053519, 20030152490, 20110160078, and U.S. Pat. Nos. 6,582,908 and 7,537,898 and 9,598,731. Tags can be linked to sample nucleic acids randomly or non-randomly.

The unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

### D. Capture Moieties

As discussed above, nucleic acids in a sample can be subject to a capture step, in which molecules having target regions are captured for subsequent analysis. Target capture can involve use of probes (e.g., oligonucleotides) labeled with a capture moiety, such as biotin, and a second moiety or binding partner that binds to the capture moiety, such as streptavidin. In some embodiments, a capture moiety and binding partner can have higher and lower capture yields for different sets of target regions, such as those of the sequence-variable target region set and the epigenetic target region set, respectively, as discussed elsewhere herein. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### E. Collections of Target-Specific Probes

In some embodiments, a collection of target-specific probes is used in a method comprising an epigenetic target region set and/or a sequence-variable target region set, as described herein. In some embodiments, the collection of target-specific probes comprises target-binding probes specific for a sequence-variable target region set and target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of target-specific probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25-to 1.5-, 1.5-to 1.75-, 1.75-to 2-, 2-to 2.25-, 2.25-to 2.5-, 2.5-to 2.75-, 2.75-to 3-, 3-to 3.5-, 3.5-to 4-, 4-to 4.5-, 4.5-to 5-, 5-to 5.5-, 5.5-to 6-, 6-to 7-, 7-to 8-, 8-to 9-, 9-to 10-, 10-to 11-, 11-to 12-, 13-to 14-, or 14-to 15-fold higher than the capture yield of the target-binding probes specific for the epigenetic target region set.

In some embodiments, the collection of target-specific probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of target-specific probes is configured to have a capture yield specific for the sequence-variable target region set is 1.25-to 1.5-, 1.5-to 1.75-, 1.75-to 2-, 2-to 2.25-, 2.25-to 2.5-, 2.5-to 2.75-, 2.75-to 3-, 3-to 3.5-, 3.5-to 4-, 4-to 4.5-, 4.5-to 5-, 5-to 5.5-, 5.5-to 6-, 6-to 7-, 7-to 8-, 8-to 9-, 9-to 10-, 10-to 11-, 11-to 12-, 13-to 14-, or 14-to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the target-specific probes specific for the sequence-variable target region set are present at a higher concentration than the target-specific probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25-to 1.5-, 1.5-to 1.75-, 1.75-to 2-, 2-to 2.25-, 2.25-to 2.5-, 2.5-to 2.75-, 2.75-to 3-, 3-to 3.5-, 3.5-to 4-, 4-to 4.5-, 4.5-to 5-, 5-to 5.5-, 5.5-to 6-, 6-to 7-, 7-to 8-, 8-to 9-, 9-to 10-, 10-to 11-, 11-to 12-, 13-to 14-, or 14-to 15-fold higher than the concentration of the target-binding probes specific for the epigenetic target region set. In such embodiments, concentration may refer to the average mass per volume concentration of individual probes in each set.

In some embodiments, the target-specific probes specific for the sequence-variable target region set have a higher affinity for their targets than the target-specific probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39:8740-8751 (2011); Freier et al., Nucleic Acids Res. 25:4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the target-specific probes specific for the sequence-variable target region set have modifications that increase their affinity for their targets. In some embodiments, alternatively or additionally, the target-specific probes specific for the epigenetic target region set have modifications that decrease their affinity for their targets. In some embodiments, the target-specific probes specific for the sequence-variable target region set have longer average lengths and/or higher average melting temperatures than the target-specific probes specific for the epigenetic target region set. These embodiments may be combined with each other and/or with differences in concentration as discussed above to achieve a desired fold difference in capture yield, such as any fold difference or range thereof described above.

In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes specific for the sequence-variable target region set and/or the target-specific probes specific for the epigenetic target region set comprise a capture moiety as discussed above, e.g., probes comprising capture moieties and sequences selected to tile across a panel of regions, such as genes.

In some embodiments, the target-specific probes are provided in a single composition. The single composition may be a solution (liquid or frozen). Alternatively, it may be a lyophilizate.

Alternatively, the target-specific probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Probes Specific for Epigenetic Target Regions

The probes for the epigenetic target region set may comprise probes specific for one or more types of target regions likely to differentiate DNA originating from different types of immune cells, including rare immune cell types, and/or to differentiate DNA from precancerous or neoplastic (e.g., tumor or cancer) cells from healthy cells, e.g., non-neoplastic circulating cells. Exemplary types of such regions are discussed in detail herein. The probes for the epigenetic target region set may also comprise probes for one or more control regions, e.g., as described herein.

In some embodiments, the probes for the epigenetic target region probe set have a footprint of at least 100 kb, e.g., at least 200 kb, at least 300 kb, or at least 400 kb. In some embodiments, the probes for the epigenetic target region set have a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb. In some embodiments, the probes for the epigenetic target region probe set have a footprint of at least 5 kb, e.g., at least 10, 20, or 50 kb.

a. Hypermethylation Variable Target Regions

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci that are differentially methylated in different immune cell types. In some embodiments, each immune cell type specific hypermethylation variable target region comprises at least one CpG site that is methylated with a frequency greater than or equal to 0.3, 0.4, 0.5, or 0.6 in one immune cell type and with a frequency less than or equal to 0.1, 0.2, or 0.3 in all other immune cell types. In some embodiments, each immune cell type specific hypermethylation variable target region comprises at least two CpG sites within 100 base pairs of each other that are each methylated with a frequency greater than or equal to 0.3, 0.4, 0.5, or 0.6 in one immune cell type and with a frequency less than or equal to 0.1, 0.2, or 0.3 in all other immune cell types. In some such embodiments, each immune cell type specific hypermethylation variable target region comprises a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency greater than 0.1, 0.2, or 0.3 in any normal tissue type. In some embodiments, each immune cell type specific epigenetic target region set comprises at least 3, at least 5, at least 10, at least 20, or at least 30 hypermethylation variable target regions that are uniquely hypermethylated in each one of the immune cell types that are identified in the method.

In some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2.

In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. The hypomethylation variable target regions may be any of those set forth above. For example, in some embodiments, the probes specific for hypomethylation variable target regions comprise probes specific for a plurality of loci that are differentially methylated in different immune cell types. In some embodiments, each immune cell type specific hypomethylation variable target region comprises at least one CpG site that is methylated with a frequency less than or equal to 0.1, 0.2, or 0.3 in one immune cell type and with a frequency greater than or equal to 0.3, 0.4, 0.5, or 0.6 in all other immune cell types. In some embodiments, each immune cell type specific hypomethylation variable target region comprises at least two CpG sites within 100 base pairs of each other that are each methylated with a frequency less than or equal to 0.1, 0.2, or 0.3 in one immune cell type and with a frequency greater than or equal to 0.3, 0.4, 0.5, or 0.6 in all other immune cell types. In some such embodiments, each immune cell type specific hypomethylation variable target region comprises a total of at least 2, 3, 4, 5, 6, 7, 8, 9, or 10 CpG sites within 150 base pairs or within 200 base pairs, wherein fewer than three of the at least 2, 3, 4, 5, 6, 7, 8, 9, or 10 CpG sites are methylated with a frequency less than 0.1, 0.2, or 0.3 in any normal tissue type. In some embodiments, each immune cell type specific epigenetic target region set comprises at least 3, at least 5, at least 10, at least 20, or at least 30 hypomethylation variable target regions that are uniquely hypomethylated in each one of the immune cell types that are identified in the method.

In some embodiments, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 2. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 3. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 3. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1.

### F. Computer Systems

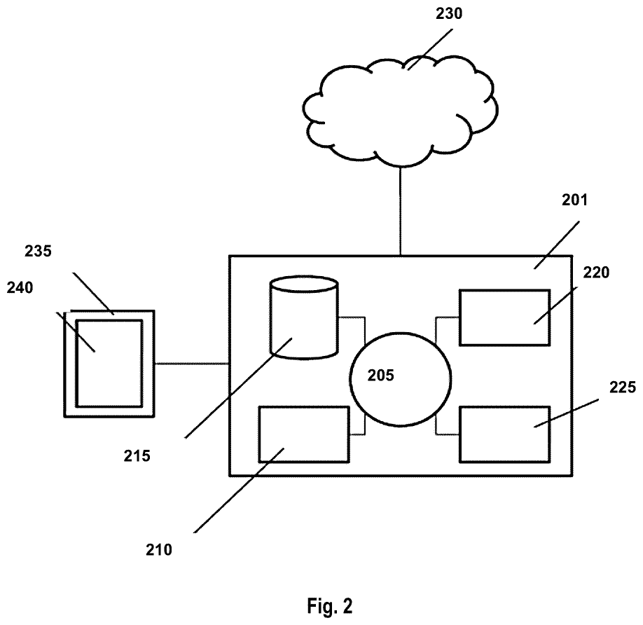

Methods of the present disclosure can be implemented using, or with the aid of, computer systems. FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 0, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method comprising: collecting cfDNA from a sample or a subject; capturing a plurality of sets of target regions from the cfDNA, wherein the plurality of target region sets comprises a sequence-variable target region set, and/or an epigenetic target region set; sequencing the captured cfDNA molecules, wherein the captured cfDNA molecules of sequence-variable target region sets are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the levels of a plurality of immune cell types and/or the likelihood that the subject has cancer.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

### G. Applications

### 1. Immune Cell Type Quantification, Cancer, and Other Diseases

The present methods can be used to quantify levels of different immune cell types, including rare immune cell types, such as activated lymphocytes and myeloid cells at particular stages of differentiation. Such quantification can be based on the numbers of molecules corresponding to a given cell type in a sample. For example, Examples 2 and 3 illustrate quantification based on hypermethylation- and hypomethylation-variable target regions, respectively. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells.

The present methods can be used to diagnose presence of conditions, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. In some embodiments, the cancer is a type of cancer that is not a hematological cancer, e.g., a solid tumor cancer such as a carcinoma or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardio facial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Subject and/or Classifying a Subject as being a Candidate for a Subsequent Cancer Treatment

In some embodiments, a method provided herein is a method of determining a risk of cancer recurrence in a subject. In some embodiments, a method provided herein is a method of classifying a subject as being a candidate for a subsequent cancer treatment.

Any of such methods may comprise collecting DNA (e.g., originating or derived from a tumor cell) from the subject diagnosed with the cancer at one or more preselected timepoints following one or more previous cancer treatments to the subject. The subject may be any of the subjects described herein. The DNA may be cfDNA. The DNA may be obtained from a tissue sample.

Any of such methods may comprise capturing a plurality of sets of target regions from DNA from the subject, wherein the plurality of target region sets comprise a sequence-variable target region set, and/or an epigenetic target region set, whereby a captured set of DNA molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of a sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises sequence-variable target region sequences and determining the cancer recurrence score may comprise determining at least a first subscore indicative of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information comprises epigenetic target region sequences, and determining the cancer recurrence score comprises determining a second subscore indicative of the amount of molecules (obtained from the epigenetic target region sequences) that represent an epigenetic state different from DNA found in a corresponding sample from a healthy subject (e.g., cfDNA found in a blood sample from a healthy subject, or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or both of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 104, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Therapies and Related Administration

In certain embodiments, the methods disclosed herein relate to identifying and administering therapies, such as customized therapies, to patients or subjects. In some embodiments, determination of the levels of particular immune cell types, including rare immune cell types, facilitates selection of appropriate treatment. In some embodiments, the patient or subject has a given disease, disorder or condition. Essentially any cancer therapy (e.g., surgical therapy, radiation therapy, chemotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone,

Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agents targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### III. Kits

Also provided are kits comprising the compositions as described herein. The kits can be useful in performing the methods as described herein. In some embodiments, a kit comprises an agent that recognizes methyl cytosine in DNA. In some such embodiments, the agent is an antibody or a methyl binding protein or methyl binding domain. In some embodiments, the kit comprises target-specific probes that specifically bind to epigenetic and/or sequence-variable target region sets, wherein the target-specific probes of at least one epigenetic target region set bind to target regions that are differentially methylated in different immune cell types. In some such embodiments, the target-specific probes comprise a capture moiety. In some embodiments, the kit comprises a solid support linked to a binding partner of the capture moiety. In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABLl, AKT1, ATM, CDHl, CSFIR, CTNNBl, ERBB4, EZH2, FGFRl, FGFR2, FGFR3, FLT3, GNA11, GNAQ, GNAS, HNFIA, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRKl. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached {e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the can be found throughout and are applicable to the kit.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Analysis of Methylation Data to Identify Differentially Methylated Regions Exclusive to Particular Immune Cell Types

Whole-genome bisulfite sequencing data from the Blueprint consortium, available on the internet at dcc.blueprint-epigenome.eu, was used in the panel design. The dataset contains methylation profiles of hundreds cell types from hematopoietic lineages, including naive and activated T and B cells. Rare immune cell types are likely to have elevated turnover in disease state and are therefore targeted in the panel, including memory B cells, plasma cells, Tregs, CD4 effector memory T cells, CD4 central memory T cells, CD8 effector memory T cells, CD8 central memory T cells, NK cells, and various myeloid cell types.

The Blueprint dataset contains single CpG level methylation level. A CpG site was considered to be detected if it is present in at least one sample, and samples that have no data for more than 10\hat{ }7 detected CpG sites were excluded from further analysis. CpG sites that are missing from at least 5 samples were filtered out. Cell types with similar results were initially grouped into clusters, including myeloid cells, naïve B cells, activated B cells, naïve T cells, activated T cells, and NK cells.

The genome was divided into 200 base pair bins with 50 base pair steps. Any bin with fewer than 7 detected CpG sites within it was excluded from downstream analysis. The methylation level of each bin in each sample was calculated as the mean of the CpG methylation level.

The mean methylation level per bin per cell cluster was calculated. A bin was identified as a differentially methylated region (DMR) for a cluster based on the extent to which the methylation level in the cluster for the DMR exceeded the maximum methylation of the region in any other cluster (hypermethylated region), or the extent to which the methylation level was less than the minimum methylation level of the other clusters (hypomethylated region). The goal was to identify 25 hypermethylated regions and 25 hypomethylated regions for each cluster. In some clusters, the goal for hypermethylated regions was not reached, while greater than 25 hypomethylated regions were readily identified; therefore, if the goal was not reached for the hypermethylated regions, additional hypomethylated regions were identified. The results are shown in the Table below. The total footprint of the panel of regions identified for clusters was 33.9 kb.

TABLE 6

| | Number of identified | Number of identified |
|---|---|---|
| Cell cluster | hypermethylated regions | hypomethylated regions |
| Naïve B cells | 23 | 27 |
| Myeloid cells | 25 | 25 |
| Naïve T cells | 18 | 32 |
| Activated B cells | 25 | 25 |
| Activated T cells | 25 | 25 |
| NK cells | 14 | 36 |

The mean methylation level per bin per cell type was then calculated. In all cell types within a cluster, DMRs that can differentiate one cell type from the other cell types within the same cluster were identified. A bin was identified as a cell type DMR based on the extent to which the methylation level in the cell type for the DMR exceeded the mean methylation of the region in any other cell types within the cluster (hypermethylated region), or the extent to which the methylation level was less than the mean methylation level of the other cell types within the cluster (hypomethylated region). The goal was to identify 25 hypermethylated regions and 25 hypomethylated regions for each cell type. The results are shown in the Table below. The total footprint of the panel of regions identified for cell types was 63.75 kb.

TABLE 7

| | Number of | Number of |
|---|---|---|
| | identified | identified |
| | hypermethylated | hypomethylated |
| Cell type | regions | regions |
| Plasma cells | 25 | 25 |
| Memory B cells | 25 | 25 |
| Naïve CD4 T cells | 25 | 25 |
| Naïve CD8 T cells | 25 | 25 |
| Regulatory T cells | 25 | 25 |
| CD4 effector memory T cells | 25 | 25 |
| CD4 central memory T cells | 25 | 25 |
| CD8 effector memory T cells | 25 | 25 |
| CD8 central memory T cells | 25 | 25 |
| Monocytes | 25 | 25 |
| Macrophages | 25 | 25 |
| Myelocytes | 25 | 25 |
| Metamyelocytes | 25 | 25 |
| Neutrophils | 25 | 25 |
| Eosinophils | 25 | 25 |
| NK cells | 14 | 36 |

Overlapped DMRs identified from bins were merged into regions, and hg38 coordinates from the Blueprint dataset were converted to hg19 coordinates using liftover (See genome.ucsc.edu/cgi-bin/hgLiftOver.) The total footprint of the resulting panel of DMRs was 95.9 kb for hg19. The results are shown in the table below.

TABLE 8

| | Number of | Number of |
|---|---|---|
| | identified | identified |
| | hypermethylated | hypomethylated |
| | regions | regions |
| Cluster | | |
| Naïve B cells | 7 | 12 |
| Myeloid cells | 8 | 13 |
| Naïve T cells | 10 | 13 |
| Activated B cells | 9 | 14 |
| Activated T cells | 7 | 12 |
| NK cells | 4 | 17 |
| Cell type | | |
| Plasma cells | 12 | 12 |
| Memory B cells | 12 | 12 |
| Naïve CD4 T cells | 8 | 13 |
| Naïve CD8 T cells | 13 | 8 |
| Regulatory T cells | 9 | 10 |
| CD4 effector memory T cells | 9 | 11 |
| CD4 central memory T cells | 9 | 6 |
| CD8 effector memory T cells | 8 | 11 |
| CD8 central memory T cells | 11 | 9 |
| Monocytes | 14 | 14 |
| Macrophages | 16 | 12 |
| Myelocytes | 12 | 16 |
| Metamyelocytes | 10 | 15 |
| Neutrophils | 12 | 16 |
| Eosinophils | 16 | 16 |
| NK cells | 4 | 17 |

Loci showing differential methylation in a cell type or cluster were plotted in a heat map showing methylation level as a function of cell type or cluster in FIG. 1B.

### Example 2: Analysis of cfDNA to Detect Hypermethylation Signal Associated with Certain Immune Cell Types for Detecting Cancer

A set of samples from healthy subjects and subjects with early-stage colorectal cancer (FIG. 1C), a type of early-stage cancer as indicated in FIG. 1D, or a type of late-stage cancer as indicated in FIG. 1E were analyzed by a blood-based assay to detect hypermethylation signal associated with certain immune cell types and test whether such signal is predictive of colorectal cancer. cfDNA was extracted from the plasma of these patients, and was then combined with MBD-coated beads to partition hypermethylated DNA from hypomethylated DNA. Any non-methylated or less methylated DNA was first eluted from the beads with buffers containing increasing concentrations of salt. Finally, a high salt buffer was used to wash the heavily methylated DNA away from the antibody specific for methyl cytosine. The unbound DNA and these washes resulted three partitions (hypomethylated, residual methylation and hypermethylated partitions) of increasingly methylated cfDNA. The cfDNA molecules in the partitions were cleaned, to remove salt, and concentrated in preparation for the enzymatic steps of library preparation.

After concentrating the cfDNA in the partitions, first adapters were added to the cfDNA by ligation to the 3′ ends thereof. The adapter was used as a priming site for second-strand synthesis using a universal primer and a DNA polymerase. The first adapter comprised a biotin, and nucleic acid ligated to the first adapter was bound to beads comprising streptavidin. A second adapter was then be ligated to the 3′ end of the second strand of the now double-stranded molecules. These adapters contained non-unique molecular barcodes and each partition was ligated with adapters having non-unique molecular barcodes that are distinguishable from the barcodes in the adapters used in the other partitions. After ligation, the hypermethylated and residual methylation partitions were treated with a methylation-sensitive restriction enzyme to degrade mispartitioned unmethylated DNA. The partitions were pooled together and were amplified by PCR.

Following PCR, amplified DNA was washed and concentrated prior to enrichment. Once concentrated, the amplified DNA was combined with a salt buffer and biotinylated RNA target-specific probes for hypermethylated DMRs in certain immune cell types used for analysis in Example 1, and this mixture was incubated overnight.

The biotinylated RNA target-specific probes (hybridized to DNA) were captured by streptavidin magnetic beads and separated from the amplified DNA that were not captured by a series of salt based washes, thereby enriching the sample. After enrichment, an aliquot of the enriched sample was sequenced using Illumina NovaSeq sequencer. The sequence reads generated by the sequencer were then analyzed using bioinformatic tools/algorithms. The molecular barcodes were used to identify unique molecules as well as for deconvolution of the sample into molecules that were differentially partitioned. The hypermethylated target region sequences were analyzed to detect methylated cfDNA molecules in regions used in Example 1. The relative methylation frequencies were determined as the total number of molecules in the methylated fraction (hypermethylated and intermediate partitions) normalized by the amount of input cfDNA. The results, shown in FIG. 1C-1E, show that a subset of randomly selected examples of the hypermethylated DMRs specific to immune cell types showed variation and separation between the CRC and healthy samples, supporting the conclusion that immune cell type-specific DMRs can be informative of cancer status.

### Example 3: Analysis of cfDNA to Detect Hypomethylation Signal Associated with Certain Immune Cell Types for Detecting Early-Stage Colorectal Cancer

A set of samples from healthy subjects and subjects with early-stage colorectal cancer were analyzed by a blood-based assay to detect hypomethylation signal associated with certain immune cell types and test whether such signal is predictive of colorectal cancer. cfDNA was extracted from the plasma of these patients, and was then contacted with MBD and partitioned into hypomethylated, intermediate, and hypermethylated partitions. The hypomethylated partition was subjected to methylation-dependent restriction enzyme (MDRE) digestion to degrade mispartitioned molecules. The cfDNA molecules in the partitions were cleaned, to remove salt, and concentrated in preparation for the enzymatic steps of library preparation. Adapters comprising molecular barcodes were ligated to the cfDNA molecules. The cfDNA molecules were enriched for regions of interest using oligonucleotides labeled with biotin, amplified, and sequenced.

The resulting molecule sequences were filtered to require cell-type specific hypomethylation (i.e., <10% methylation in one cell type of interest and >90% methylation in other cell types) (according to published data such as Loyfer et al., 2022, doi.org/10.1101/2022.01.24.477547; Fox-Fisher et al., eLife 2021, 10: e70520; Moss et al., Nature Communications 2021, 9:5068; and the EpiDISH R package available at www.bioconductor.org/packages/release/bioc/html/EpiDISH.html), the presence of at least one MDRE restriction site, The cell types of interest were B cells, granulocytes, NK cells, T cells, and erythrocyte progenitors. Sites having low molecular coverage were also filtered out.

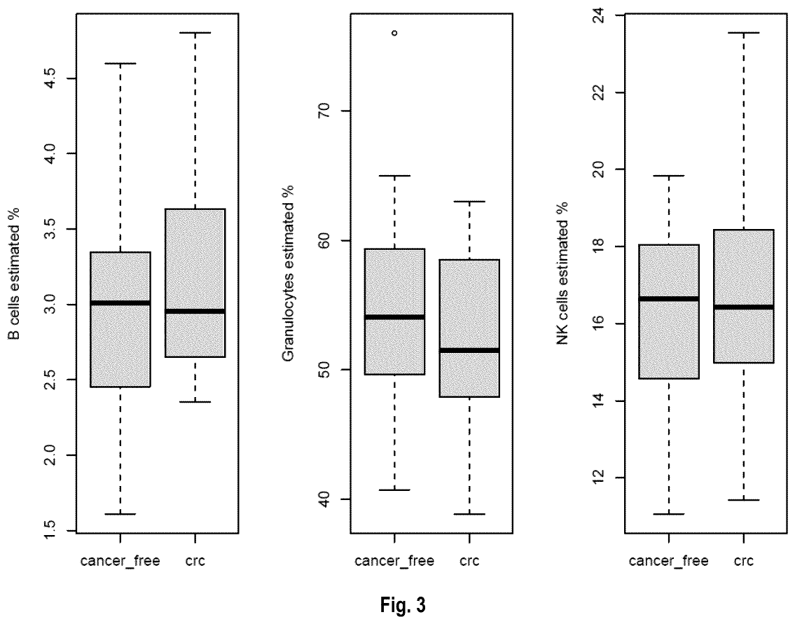

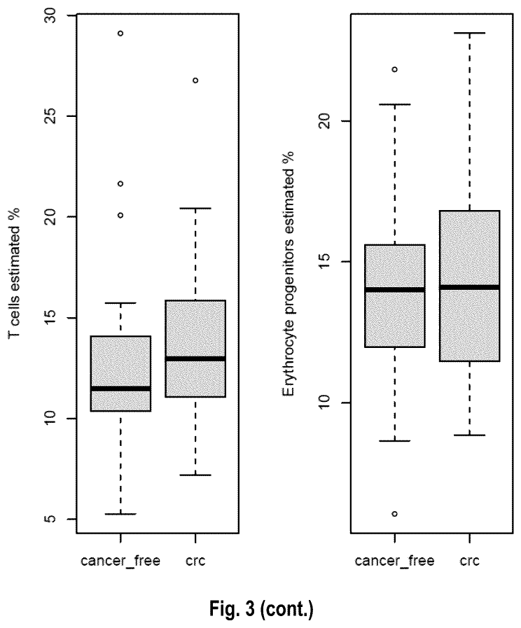

The fraction of cfDNA contributed by each cell type of interest was determined as follows. For each site that shows hypomethylation specific for that cell type, the proportion of hypomethylated DNA was determined, and these values were averaged to give the fraction of cfDNA contributed by the cell type. Results are shown in FIG. 3 and are consistent with higher contributions from B cells, NK cells, and T cells, and lower contributions from granulocytes, to cfDNA from subjects having CRC than healthy subjects.

### Example 4: Analysis of cfDNA to Quantify Immune Cell Types and to Detect the Presence or Absence of Cancer in a Subject

A set of samples from healthy subjects, subjects with early-stage colorectal cancer, and cancer patients are analyzed by a blood-based assay to detect levels of immune cell types and the presence/absence of mutations. cfDNA is extracted from the plasma of these patients and is then combined with an antibody specific for methyl cytosine. Magnetic beads conjugated with protein G are used to immunoprecipitate the antibody and DNA bound thereto, thus partitioning hypermethylated DNA from hypomethylated DNA. Any non-methylated or less methylated DNA is first eluted from the beads with buffers containing increasing concentrations of salt. Finally, a high salt buffer is used to wash the heavily methylated DNA away from the antibody specific for methyl cytosine. The unbound DNA and these washes result in at least three partitions (hypomethylated, residual methylation and hypermethylated partitions) of increasingly methylated cfDNA. The cfDNA molecules in the partitions are cleaned, to remove salt, and concentrated in preparation for the enzymatic steps of library preparation.

After concentrating the cfDNA in the partitions, first adapters are added to the cfDNA by ligation to the 3′ ends thereof. The adapter is used as a priming site for second-strand synthesis using a universal primer and a DNA polymerase. The first adapter comprises a biotin, and nucleic acid ligated to the first adapter is bound to beads comprising streptavidin. A second adapter is then be ligated to the 3′ end of the second strand of the now double-stranded molecules. These adapters contain non-unique molecular barcodes and each partition is ligated with adapters having non-unique molecular barcodes that is distinguishable from the barcodes in the adapters used in the other partitions. After ligation, the partitions are pooled together and are amplified by PCR.

Following PCR, amplified DNA is washed and concentrated prior to enrichment. Once concentrated, the amplified DNA is combined with a salt buffer and biotinylated RNA target-specific probes that comprise probes for a sequence-variable target region set and probes for an epigenetic target region set and this mixture is incubated overnight. The probes for the sequence-variable region set has a footprint of about 50 kb and the probes for the epigenetic target region set has a footprint of about 500 kb. The probes for the sequence-variable target region set comprise oligonucleotides targeting at least a subset of genes identified in Tables 3-5 and the probes for the epigenetic target region set comprises oligonucleotides targeting a selection including at least hypermethylation variable target regions and hypomethylation variable target regions, and optionally including CTCF binding target regions, transcription start site target regions, focal amplification target regions and methylation control regions. The hypermethylation variable target regions and hypomethylation variable target regions include regions specific for a plurality of clusters and/or immune cell types selected from those listed in Table 8.

The biotinylated RNA target-specific probes (hybridized to DNA) are captured by streptavidin magnetic beads and separated from the amplified DNA that are not captured by a series of salt based washes, thereby enriching the sample. After enrichment, an aliquot of the enriched sample is sequenced using Illumina NovaSeq sequencer. The sequence reads generated by the sequencer are then analyzed using bioinformatic tools/algorithms. The molecular barcodes are used to identify unique molecules as well as for deconvolution of the sample into molecules that were differentially partitioned. The sequence-variable target region sequences are analyzed by detecting genomic alterations such as SNVs, insertions, deletions and fusions that can be called with enough support that differentiates real tumor variants from technical errors (for e.g., PCR errors, sequencing errors). The epigenetic target region sequences are analyzed independently to detect methylated cfDNA molecules in regions that have been shown to be differentially methylated in different immune cells and in cancer compared to normal cells. Finally, the results of both analyses are combined to produce a final determination of the likelihood of cancer or precancer in the subjects from which the samples are obtained.

1.-103. (canceled)

104. A method of analyzing DNA in a sample, the method comprising:
a) partitioning the sample into a plurality of subsamples by contacting the DNA with an agent that recognizes a modified cytosine in the DNA, the plurality comprising a first subsample and a second subsample, wherein the first subsample comprises DNA with the modified cytosine in a greater proportion than the second subsample;
b) capturing at least an epigenetic target region set of DNA from at least one of the first and second subsamples, comprising contacting the DNA with target-specific probes specific for the at least one epigenetic target region set, wherein the target regions of the epigenetic target region set comprise DNA sequences that are differentially methylated in a plurality of immune cell types, wherein the plurality of immune cell types comprises
i. naïve and activated lymphocytes;
ii. monocytes and macrophages; or
iii. myelocytes, neutrophils, and eosinophils,
thereby providing captured DNA; and
c) sequencing the captured DNA and determining levels of each of a plurality of immune cell types from which the DNA originated.

105. The method of claim 104, wherein the partitioning comprises contacting the DNA of the sample with a methyl binding reagent that specifically recognizes 5-methylcytosine.

106. The method of claim 104, wherein the method comprises capturing at least an epigenetic target region set of DNA from at least one of the first and second subsamples, wherein the target regions of the epigenetic target region set comprise DNA sequences that are differentially methylated in a plurality of immune cell types.

107. The method of claim 106, comprising contacting at least one subsample with a restriction enzyme prior to the capturing or sequencing, wherein the contacting occurs after partitioning the sample into the plurality of subsamples.

108. The method of claim 107, wherein the restriction enzyme is a MDRE.

109. The method of claim 108, wherein the second subsample is contacted with the MDRE.

110. The method of claim 109, wherein the epigenetic target region set comprises hypomethylation variable target regions comprising DNA sequences that are differentially hypomethylated in a plurality of immune cell types.

111. The method of claim 110, wherein the DNA sequences that are differentially hypomethylated in a plurality of immune cell types comprise a detectably lower degree of methylation in at least one immune cell type than the degree of methylation of the same sequence in any other immune cell type.

112. The method of claim 111, wherein the detectably lower degree of methylation is three fewer methylated cytosines than the same sequence in the any other immune cell type.

113. The method of claim 107, wherein the restriction enzyme is a MSRE.

114. The method of claim 113, wherein the first subsample is contacted with the MSRE.

115. The method of claim 114, wherein the epigenetic target region set comprises hypermethylation variable target regions comprising DNA sequences that are differentially hypermethylated in a plurality of immune cell types.

116. The method of claim 115, wherein the DNA sequences that are differentially hypermethylated in a plurality of immune cell types comprise a detectably higher degree of methylation in at least one immune cell type than the degree of methylation of the same sequence in any other immune cell type.

117. The method of claim 116, wherein the detectably higher degree of methylation is three more cytosine methylations than the same sequence in the any other immune cell type.

118. The method of claim 104, wherein the method comprises ligating adapters to the DNA, thereby producing adapter-ligated DNA, wherein the adapter-ligated DNA is amplified prior to the sequencing.

119. The method of claim 119, wherein the subsamples are pooled prior to the sequencing.

120. The method of claim 104, wherein the plurality of immune cell types comprises metamyelocytes and/or natural killer (NK) cells.

121. The method of claim 104, wherein the levels of each of the plurality of immune cell types are determined relative to levels of total blood cells.

122. The method of claim 104, wherein the DNA comprises cell free DNA (cfDNA).

123. The method of claim 104, comprising determining a ratio of levels or quantities of immune cells types based on the determined levels or quantities of the plurality of immune cell types.
